(19) United States
(12) Patent Application Publication
(10) Pub. No.: US 2023/0242905 A1
(43) Pub. Date: Aug. 3, 2023

(54) Method for Engineering Novel Hybrid AAV Capsids Through Hyper Variable Regions Swapping

(71) Applicants: Genethon, Evry-Courcouronnes (FR); Institut National de la Santé et de la Recherche Médicale, Paris (FR); Universite d'Evry Val d'Essonne, Evry (FR)

(72) Inventors: Giuseppe Ronzitti, Fontainebleau (FR); Tiziana Labella, Paris (FR)

(73) Assignees: Genethon, Evry-Courcouronnes (FR); Institut National de la Santé et de la Recherche Médicale, Paris (FR); Universite d'Evry Val d'Essonne, Evry (FR)

(21) Appl. No.: 18/013,339

(22) PCT Filed: Jul. 5, 2021
(86) PCT No.: PCT/EP2021/068553

§ 371 Date: Dec. 28, 2022

(30) Foreign Application Priority Data
Jul. 3, 2020 (EP) 20305757.5

(51) Int. Cl. C12N 15/10; C12N 15/86

(52) U.S. Cl. CPC C12N 15/1082; C12N 15/86; C12N 2750/14122; C12N 2750/14143; C12N 2750/14145; C12N 2750/14152

(57) ABSTRACT

The invention relates to a method of preparation of a recombinant hybrid adeno-associated virus (AAV) capsid protein with improved tropism and to the recombinant hybrid AAV capsid protein obtainable by the method. The invention relates also to the derived expression vector, modified cell, and hybrid capsid AAV vector particle packaging a gene of interest, and its use in tissue-targeted gene therapy for treating various diseases.

### FIELD OF THE INVENTION

The invention relates to a method of preparation of a recombinant hybrid adeno-associated virus (AAV) capsid protein with improved tropism, in particular for muscle and/or central nervous system, and to the recombinant hybrid AAV capsid protein obtainable by the method. The invention relates also to the derived expression vector, modified cell, and hybrid capsid AAV vector particle packaging a gene of interest, and its use in tissue-targeted gene therapy for treating various diseases, in particular muscle and/or central nervous system diseases.

### BACKGROUND OF THE INVENTION

Recombinant AAV (rAAV) vectors represent the leading platform for gene therapy in a wide spectrum of organs for the treatment of a variety of human diseases. The exponential growth of clinical trials using rAAV reflects the enormous potential of this system and its high versatility (Valdmanis P N et al., Hum. Gene Ther., 2017, 28, 361-372; Wang D et al., Nat. Rev. Drug Discov., 2019, 18, 358-378).

AAV is a non-pathogenic virus belonging to the genus Dependoparvovirus within the family Parvoviridae. AAV is a non-enveloped virus composed of a capsid of about 26 nm in diameter and a single-stranded DNA genome of 4.7 kb. The genome carries two genes, rep and cap, flanked by two palindromic regions named Inverted terminal Repeats (ITR) that serve as the viral origins of replication and the packaging signal. The cap gene codes for three structural proteins VP1, VP2 and VP3 that compose the AAV capsid through alternative splicing and translation from different start codons. VP1, VP2 and VP3 share the same C-terminal end which is all of VP3. Using AAV2 has a reference, VP1 has a 735 amino acid sequence (GenBank accession number YP_680426.1 accessed on 13 Aug. 2018); VP2 (598 amino acids) starts at the Threonine 138 (T138) and VP3 (533 amino acids) starts at the methionine 203 (M203). The rep gene encodes four proteins required for viral replication Rep78, Rep68, Rep52 and Rep40. Recombinant AAV vectors encapsidate an ITR-flanked rAAV genome in which a therapeutic gene expression cassette replaces the AAV protein coding-sequences.

The development of an efficacious AAV platform is the result of a synergic approach between capsid and vector genome design. In this context, the capsid plays a crucial role in tissue targeting through its interaction with cellular receptors and the following downstream internalization events. Tissue tropism and transduction efficiency are directly linked to the sequence and conformation of the looped-out domains of the VP proteins that compose the capsids. Noteworthy, the amino acid variability of VP sequence of different AAV serotypes clusters in 12 hypervariable regions (HVR) which mainly corresponds to the looped-out domains (Gao G et al., Proc Natl Acad Sci USA., 2003, 100, 6081-6086).

Strategies for developing new capsids can be categorized in four main approaches: natural discovery, rational design, directed evolution and in silico discovery (Wang D et al. Nat. Rev. Drug Discov. 2019, 100, 6081-6086). Natural discovery consists in the isolation of wild type AAV that naturally infect animals, including human and non-human primate. Notably, AAV isolated from human sources, such as AAV9, are the most promising serotypes (Gao G et al., J Virol., 2004, 78, 6381-6388).

Rational design strategy mainly involves the grafting of peptide that confer new properties to the capsid, like increase the receptor binding or deter immunological recognition (Chen Y H et al., Nat. Med., 2009, 15, 1215-1218; Asokan A et al., Nat. Biotechnol., 2010, 28, 79-82).

Direct evolution approach simulates the natural evolution. Basically, by using error-prone PCR or capsids shuffling strategies a library of randomized capsids is generated and submitted to selective pressure in order to select capsids with specific properties (Wang D et al., Nat. Rev. Drug Discov., 2019, 18, 358-378). Finally, with advancement of the high-throughput sequencing, the bioinformatics met the field of capsid development, this approach is named in silico discovery. Bioinformatic tools can be used to predict the capsids regions that better tolerate manipulation, or to infer evolutionary intermediated of known capsid, an approach exemplified by the discovery of the ancestral capsid Anc80 (Marsic, D. et al., Mol. Ther., 2014, 22, 1900-1909; Zinn E et al., Cell Rep., 2015, 12, 1056-1068).

However, each approach has specific limitations that may affect the transduction efficiency of rAAV (Wang D et al. Nat. Rev. Drug Discov., 2019, 18, 358-378). First, AAV infection is endemic in human population, therefore the rAAV has to face the pre-existing capsid immunity, especially when using capsid isolated from human sources (Boutin et al., Human Gene Therapy, 2010, June; 21(6):704-12. doi: 10.1089/hum.2009.182). Rational design approach can help to overcome this problem, however the insufficient knowledge on the stability of modified capsid, AAV receptor binding, internalization and cellular trafficking poses a major limitation to this strategy. In addition, the choice of the animal model is crucial to proper select novel capsids with the best performances for gene therapy application in human. This is particularly true when using an approach of direct evolution, where capsids selection is deeply rooted by the model system.

To improve AAV vectors used in gene therapy, there is a need for novel AAV capsid engineering strategies that at least partly overcome the limitations of existing approaches.

### SUMMARY OF THE INVENTION

The inventors have shown that combination of hypervariable regions (HVRs) from different AAV serotypes can result in mixed features of parental capsids outperforming their original efficacies. In particular, the inventors have obtained hybrid AAV capsids which advantageously improve the tropism compared to at least the parent acceptor capsid and at the same time maintain the low seroprevalence of the acceptor capsid. This is surprising since the sequence and conformation of the 12 HVRs are directly involved in both the tropism and seroprevalence of AAV capsids, the molecular determinants of which remain to be fully elucidated. Therefore, it is unexpected to improve the tropism without impairing the seroprevalence.

Therefore, the invention relates to a method of preparation of a recombinant hybrid adeno-associated virus (AAV) capsid protein with improved tropism for muscle and/or central nervous system, comprising the steps of
a) providing at least two recombinant AAV capsid proteins from different AAV serotypes, an acceptor AAV capsid protein and at least one donor AAV capsid protein; wherein the donor AAV capsid serotype is AAV13 or hybrid AAV2/13;
b) replacing at least one hypervariable region (HVR) sequence chosen from HVR1 to HVR10 and HVR12 sequences of the acceptor AAV capsid protein with a different HVR sequence from the corresponding HVR of a donor AAV capsid protein, to obtain a recombinant hybrid AAV capsid protein with improved tropism for muscle and/or central nervous system compared to at least the parent acceptor AAV capsid protein.

In some embodiments of the method according to the invention, the acceptor AAV capsid serotype has a low seroprevalence and the donor AAV capsid serotype(s) has a higher seroprevalence than the acceptor AAV capsid serotype. In some preferred embodiments of the method according to the invention, the hybrid AAV capsid protein has a seroprevalence equivalent to the seroprevalence of the acceptor AAV capsid protein.

In some preferred embodiments of the method according to the invention, the acceptor AAV capsid serotype is selected from the group consisting of: AAV8, AAV9, AAV5, AAVrh10, AAV-LK03, AAVrh74, AAV9.rh74, AAV9.rh74-P1 and/or the donor AAV capsid serotype(s) is selected from the group consisting of AAV13, and the sequences SEQ ID NO: 2 to 30.

In some embodiments of the method according to the invention, the HVR sequence(s) of the donor AAV capsid protein and/or acceptor AAV capsid protein(s) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1.

In some embodiments of the method according to the invention, step b) comprises replacing less than 8 HVR sequences of the acceptor AAV capsid protein with different HVR sequence(s) from the corresponding HVR(s) of the donor AAV capsid protein(s); preferably step b) comprises replacing up to 6 HVR sequences, preferably up to 4 HVR sequences, of the acceptor AAV capsid protein with different HVR sequence(s) from the corresponding HVR(s) of the donor AAV capsid protein(s).

In some preferred embodiments of the method according to the invention, step b) comprises replacing at least HVR5 sequence of the acceptor AAV capsid protein with a different HVR5 sequence from the donor AAV capsid protein; preferably the HVR5 sequence from the donor AAV capsid protein comprises a sequence selected from the group consisting of SEQ ID NO: 175 to 186; preferably step b) comprises replacing HVR5 sequence alone or in combination with one or more or all of HVR6, HVR7, HVR8, HVR9 and HVR10 of the acceptor AAV capsid protein; preferably step b) comprises replacing HVR5 sequence alone or in combination with one or more or all of HVR6, HVR7 and HVR8 of the acceptor AAV capsid protein.

In some embodiments of the method according to the invention, step b) comprises replacing all of HVR5 to HVR10 sequences of the acceptor AAV capsid protein with different HVR sequence(s) from the corresponding HVR(s) of the donor AAV capsid protein(s); preferably step b) comprises replacing all of HVR5 to HVR8 sequences of the acceptor AAV capsid protein with different HVR sequence(s) from the corresponding HVR(s) of the donor AAV capsid protein(s).

In some embodiments of the method according to the invention, step b) comprises replacing any one of HVR1 to HVR10, and HVR12 sequence of the acceptor AAV capsid protein with a different HVR sequence from the corresponding HVR of the donor AAV capsid protein; preferably step b) comprises replacing HVR3, HVR5, HVR9, HVR10 or HVR12 sequence of the acceptor AAV capsid protein with a different HVR sequence from the corresponding HVR from the donor AAV capsid protein. In some more preferred embodiment, step b) comprises replacing HVR5 of the acceptor AAV capsid protein with a different HVR5 sequence from the donor AAV capsid protein.

Another aspect of the invention relates to a recombinant hybrid AAV capsid protein with improved tropism obtainable by the method according to the present disclosure.

In some particular embodiments, the recombinant hybrid AAV capsid protein comprises an amino acid sequence selected from the group consisting of the sequences SEQ ID NO: 33 to 43, 45, 47 to 58 and 60 to 73 and the sequences having at least 85% identity with said sequences, and wherein the amino acid sequence variant has no mutations in at least the HVR sequences from the donor AAV capsid protein or all the HVR sequences.

Another aspect of the invention relates to a recombinant plasmid comprising a polynucleotide encoding the recombinant hybrid AAV capsid protein according to the present disclosure in expressible form; preferably selected from the nucleotide sequences SEQ ID NO: 78, 80, 82, 84, 86, 88, 90, 92, 94, 96, 98, 102, 106, 108, 110, 112, 114, 116, 118, 120, 122, 124, 126, 128, 132, 134, 136, 138, 140, 142, 144, 146, 148, 150, 152, 154, 156, 158, and eventually further encoding AAV Replicase protein in expressible form.

Another aspect of the invention relates to a cell stably transformed with a recombinant plasmid according to the present disclosure.

Another aspect of the invention relates to an AAV vector particle packaging a gene of interest, which comprises at least one hybrid recombinant AAV capsid protein according to the present disclosure; preferably wherein the gene of interest is selected from the group consisting of: therapeutic genes; genes encoding therapeutic proteins or peptides such as therapeutic antibodies or antibody fragments and genome editing enzymes; and genes encoding therapeutic RNAs such as interfering RNAs, guide RNAs for genome editing and antisense RNAs capable of exon skipping.

Another aspect of the invention relates to a pharmaceutical composition comprising a therapeutically effective amount of AAV vector particle according to the present disclosure or cell stably transduced by said AAV vector particle. The invention also encompasses the AAV vector particle, cell or pharmaceutical composition of the present disclosure as a medicament, in particular for use in the treatment of a muscle and/or CNS disease, preferably a genetic neuromuscular disease.

### DETAILED DESCRIPTION OF THE INVENTION

### Method of Preparation of Hybrid AAV Capsids

Therefore, the invention relates to a method of preparation of a recombinant hybrid adeno-associated virus (AAV) capsid protein with improved tropism, in particular for muscle and/or CNS, comprising the steps of
a) providing at least two recombinant AAV capsid proteins from different AAV serotypes, an acceptor AAV capsid protein and at least one donor AAV capsid protein;
b) replacing at least one hypervariable region (HVR) sequence of the acceptor AAV capsid protein with a different HVR sequence from the corresponding HVR of a donor AAV capsid protein, to obtain a recombinant hybrid AAV capsid protein with improved tropism, in particular for muscle and/or CNS, compared to at least the parent acceptor AAV capsid protein(s).

As used herein “AAV serotype” or “AAV capsid serotype” refers to an AAV capsid having distinct hypervariable region (HVR) amino acid sequences compared to an AAV capsid of another serotype. Different AAV serotypes have amino acid variation in their HVR sequences. The term AAV serotype encompasses any natural or artificial AAV capsid serotype including AAV capsid variants isolated from primate (human or non-human) or non-primate species and AAV capsid variants engineered by various techniques known in the art such as for example rational design, directed evolution and in silico discovery. As used herein, the term AAV serotype refers to a functional AAV capsid which is able to form recombinant AAV viral particles which transduce a cell, tissue or organ, in particular a cell tissue or organ of interest (target cell, tissue or organ) and express a transgene in said cell, tissue or organ, in particular target cell tissue or organ.

As used herein, “hypervariable region or HVR” refers to any one of HVR1 to HVR12 of an AAV capsid. According to a narrow definition of HVR, HVR1 is from positions 146 to 153; HVR2 from positions 183-187; HVR3 from positions 263 to 267; HVR4 from positions 384 to 386; HVR5 from positions 453 to 477; HVR6 from positions 493 to 498; HVR7 from positions 503 to 507; HVR8 is from positions 517 to 525; HVR9 from positions 536 to 559; HVR10 from positions 584 to 597; HVR11 from positions 661 to 670; and HVR12 from positions 708 to 722; the indicated positions being determined by alignment with SEQ ID NO: 1 (VP1 of AAV8 or AAV8 capsid). After sequence alignment of any other AAV capsid sequence of any other serotype with SEQ ID NO: 1 using standard protein sequence alignment programs that are well-known in the art, such as for example BLAST, FASTA, CLUSTALW, MEGA and the like, a person skilled in the art can easily obtained the corresponding positions of the hypervariable regions in other AAV capsid serotypes. For example using MEGA software (version X) with ClustalW alignment algorithm at default parameters, HVR1 to HVR12 are from positions 146 to 152, 182 to 186, 262 to 264, 381 to 383, 450 to 474, 490 to 495, 500 to 504, 514 to 522, 533 to 556, 581 to 594, 658 to 667 and 705 to 719, respectively of the capsid of SEQ ID NO: 2 (named #704).

The positions of the HVR sequence from the donor or acceptor AAV capsids may differ from the positions indicated above (HVR reference sequence) by few amino acids. Depending on the initial size of the HVR and the distance between the different HVRs, both HVR sequences (replaced sequence from the acceptor capsid and replacement sequence from the donor capsid(s)) consist of at least 2 amino acids to about 70 amino acids. For example the HVR sequence from the donor or acceptor AAV capsids may have a deletion of 1 amino acid at one end of a HVR sequence of up to 5 amino acids; a deletion of up to 2 amino acids (1 or 2 amino acids) at one or both ends of a HVR sequence of 6 to 10 amino acids; a deletion of up to 5 amino acids (1, 2, 3, 4 or 5 amino acids) at one or both ends of a HVR sequence of 11 to 25 amino acids. Alternatively, the HVR sequence from the donor or acceptor AAV capsid may have additional sequence from the N- or C-terminus of the HVR sequence, for example up to 10, 20, 30, 40 or 50 amino acids from the N- or C-terminus of the HVR sequence. Preferably, the amino acid deletion or addition at one or both ends of the HVR sequence involves consecutive amino acids from the donor or acceptor AAV capsid sequence.

As used herein, the term “tropism” refers to the capacity of an AAV capsid protein present in a recombinant AAV viral particle, to transduce some particular type(s) of cell(s), tissue(s) or organ(s) (e.g, cellular or tissue tropism). The tropism of the recombinant hybrid AAV capsid protein (or hybrid AAV capsid) according to the invention for a particular type of cell, tissue or organ may be determined by measuring the ability of AAV vector particles comprising the hybrid AAV capsid protein (hybrid capsid serotype AAV vector particles) to transduce said particular type of cell, tissue or organ or express a transgene in said particular type of cell, tissue or organ, using standard assays that are well-known in the art such as those disclosed in the examples of the present application. For example, vector transduction or transgene expression are determined by local or systemic administration of hybrid capsid serotype AAV vector particles in animal models such as mouse models that are well known in the art and disclosed in the examples of the present application. Parent AAV vector serotypes comprising the donor or acceptor capsids are used for comparison. Vector transduction may be determined by measuring vector genome copy number per diploid genome by standard assays that are well known in the art such as real-time PCR assay. Transgene expression is advantageously measured using a reporter gene such as luciferase or fluorescent protein (GFP or others) by standard assays that are well known in the art such as in vivo or in vitro quantitative bioluminescence or fluorescence assays in vivo or in vitro.

The hybrid AAV capsid protein is a functional AAV capsid which is able to form recombinant AAV viral particles which transduce a cell, tissue or organ, in particular a cell tissue or organ of interest (target cell, tissue or organ) and express a transgene in said cell, tissue or organ, in particular target cell tissue or organ. Furthermore, the hybrid AAV capsid protein has improved tropism compared to its parent AAV capsid protein(s). The hybrid AAV capsid protein which has improved tropism may have an increased tropism for at least one target cell, tissue or organ and/or a decreased tropism (or detargeting) for at least one off-target cell, tissue or organ compared to at least the parent acceptor AAV capsid. An increased tropism refers in particular to a transgene expression level that is increased by at least 1.5 fold, preferably 2, 3, 4, 5 folds or more in at least one target cell, tissue or organ, compared to parent AAV capsid protein(s). A detargeting refers in particular to a transgene expression level that is decreased by at least 3 fold, preferably 5 to 10 folds or more in at least one off-target cell, tissue or organ, compared to a non-detargeted parent AAV capsid protein. The transgene expression levels achieved with the hybrid AAV capsid protein in the target cell, tissue or organ is advantageously at least of the same magnitude (less than 1.5 fold lower; i.e equivalent to) as that of a reference AAV serotype such as AAV9 for muscle and CNS tissues. As a results of its improved tropism, the hybrid AAV capsid protein according to the invention has an improved biodistribution. This means that it targets significantly better a defined tissue (target tissue), group of tissues (for example skeletal muscle and heart) or organ (target tissue(s) or organ) without increasing the targeting of other (non-target) tissues (e.g. improved specificity) and/or it targets a specific tissue (non-target or off-target tissue or organ) with a lower efficacy (tissue detargeting, for example liver detargeting), usually to reduce unwanted toxicities.

As used herein, the term “muscle” refers to cardiac muscle (i.e. heart) and skeletal muscle. The term “muscle cells” refers to myocytes, myotubes, myoblasts, and/or satellite cells. The skeletal muscles are classified in different groups based on their anatomical position in the body. The tropism of the hybrid AAV capsid according to the invention for different muscle groups may be measured in mice Tibialis (TA), Extensor Digitorum Longus (EDL), Quadriceps (Qua), Gastrocnemius (Ga), Soleus (Sol), Triceps, Biceps and/or Diaphragm; in particular in mice Extensor Digitorum Longus (EDL), Soleus (Sol), Quadriceps (Qua), Triceps and Diaphragm or Soleus (Sol), Quadriceps (Qua), Triceps and Diaphragm muscles.

As used herein, the term “central nervous system or CNS” refers to the brain, spinal cord, retina, optic nerve, and/or olfactory nerves and epithelium. As used herein, the term CNS cells refer to any cells of the CNS including neurons and glial cells (oligodendrocytes, astrocytes, ependymal cells, microglia).

As used herein “seroprevalence” refers to the human seroprevalence, which means the level of anti-AAV antibodies binding to an AAV capsid serotype present in a human population and expressed as seric antibodies or immunoglobulins. The seroprevalence of an AAV capsid is measured using a cohort of human sera and standard assays that are well known in the art and disclosed for example in (Meliani et al., Hum Gene Ther Methods. 2015 April; 26(2):45-53. doi: 10.1089/hgtb.2015.037). The assay may be an ELISA assay as disclosed in the examples of the present application. The seroprevalence of an AAV capsid serotype (or serotype) may be defined as the percentage of individuals having an ELISA titer of IgG specific for said serotype higher than 10 μg/mL. A low prevalent serotype may be defined as a serotype with less than around 30% of individuals that are seropositive, corresponding to a seroprevalence similar or lower to AAV8 capsid (SEQ ID NO: 1) seroprevalence which is considered as a reference of low-seroprevalence. A high-seroprevalent AAV capsid serotype refers to a AAV capsid serotype having a seroprevalence higher than 50%. A seroprevalence equivalent to the seroprevalence of the acceptor AAV capsid refers to a seroprevalence which is around 30%. Alternatively, the seroprevalence may be defined as the dilution at which a reduction of 50% of the OD signal is observed (OD50) using a dose-response curve. The OD50 of the tested AAV capsid is compared to that of a reference AAV capsid of known seroprevalence.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

The term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wis.) pileup program, or any of sequence comparison algorithms such as BLAST, FASTA or CLUSTALW.

The acceptor and donor AAV capsids may be from any different natural or artificial AAV serotypes. At least 13 different AAV serotypes (AAV1 to 13) have been identified in human and non-human primates and classified in various clades and clones based on phylogenetic analysis of VP1 sequences of various primate AAV isolates: AAV1 and AAV6 correspond to Clade A; AAV2 to Clade B; AAV2-AAV3 hybrid to Clade C; AAV7 to Clade D; AAV8 to Clade E; AAV9 to Clade F, whereas AAV3, AAV4 and AAV5 are disclosed as clones (Gao et al., J. Virol., 2004, 78, 6381-6388). AAV2 variant serotypes and AAV2/13 hybrid capsids have been isolated in human liver (La Bella et al., Gut, 2020, 69, 737-747.doi:10.1136/gutjnk-2019-318281; SEQ ID NO: 2 to 30 in the attached sequence listing). Other AAV serotypes have been identified in non-primate species, such as porcine, bovine, avian and caprine. Porcine AAV includes in particular AAVpo1, po2.1, po4 to 6. Various AAV capsid variants, also named “synthetic AAV serotypes” or new AAV serotypes” have been engineered, in particular by directed gene evolution or in silico discovery such as with no limitations recombinant AAV2-derived serotypes DJ, DJ8 and PHP.B which are hybrid capsids from 8 AAV serotypes (AAV2, 4, 5, 8, 9, avian, bovine and goat) AAV-Anc80, AAV2i8, AAV-LK03 and others.

In some embodiments, the acceptor AAV capsid protein is from an AAV serotype used in gene therapy, also named “conventional AAV serotype” such as for example AAV1, AAV2, AAV2 variants (such as the quadruple-mutant capsid optimized AAV2 comprising an engineered capsid with Y44+500+730F+T491V changes, disclosed in Ling et al., 2016 Jul. 18, Hum Gene Ther Methods.), AAV3 and AAV3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, AAV4, AAV5, AAV6 and AAV6 variants (such as the AAV6 variant comprising the triply mutated AAV6 capsid Y731F/Y705F/T492V form disclosed in Rosario et al., 2016, Mol Ther Methods Clin Dev. 3, p. 16026), AAV7, AAV8, AAV9, AAV 2G9, AAV10 such as AAVcyl0 and AAVrh10, AAVrh32.33, AAVrh39, AAVrh43, AAVrh74, AAV-DJ, AAVAnc80, AAV-LK03, AAV.PHP such as AAV-PHP.B, AAV-PHP.EB, AAV2i8, clade F AAVHSC such as AAVHSC7, AAVHSC15 and AAVHSC17, AAV9.rh74 and AAV9.rh74-P1 (WO 2019/193119), porcine AAV such as AAVpo1, AAVpo2.1, AAVpo4 and AAVpo6, and tyrosine, lysine and serine capsid mutants of AAV serotypes.

In particular embodiments, the acceptor AAV capsid protein is from an AAV serotype selected from the group consisting of: AAV4, AAV5, AAV7, AAV8, AAV9, AAVrh10, AAVrh32.33, AAVrh39, AAVrh43, AAVrh74, AAV9.rh74, AAV9.rh74-P1, AAV-DJ, AAVAnc80, AAV2i8, AAV-LK03, and AAV.PHP. AAV4 capsid (GenBank accession number NC_001829.1); AAV5 capsid (GenBank accession number NC_006152.1 accessed on 13 Aug. 2018); AAV7 capsid (GenBank accession number NC_006260.1); AAV9 capsid (GenBank accession number AY530579.1 accessed on 24 Jun. 2004); AAVrh10 capsid (GenBank accession number AY243015.1 accessed on 14 May 2003); AAV-LK03 (amino acid sequence SEQ ID NO: 166), AAVrh74 (amino acid sequence SEQ ID NO: 160; CDS of SEQ ID NO: 161) AAV9.rh74 (amino acid sequence SEQ ID NO: 162; CDS of SEQ ID NO: 163), AAV9.rh74-P1 (amino acid sequence SEQ ID NO: 164; CDS of SEQ ID NO: 165).

In particular embodiments, the donor AAV capsid protein(s) is from a newly-isolated natural AAV variant serotype such as for example AAV2/13 hybrid serotype, in particular isolated from human tissue such as liver tissue; more preferably selected from the group consisting of the sequences SEQ ID NO: 2 to 30. In some preferred embodiments, the donor AAV capsid protein(s) is selected from the group consisting of the sequences SEQ ID NO: 2 to 10, 18, 20-22, 29 and 30; still more preferably SEQ ID NO: 2, 10, 20, 21 and 30.

In particular embodiments, the donor AAV capsid protein(s) is from an AAV serotype used in gene therapy. The donor AAV capsid protein(s) may be AAV13. AAV13 capsid gene (coding sequence or CDS) sequence corresponds to positions 1948 to 4149 of AAV13 genome sequence GenBank accession number EU285562.1 as accessed on 23 September; AAV13 capsid protein (major coat protein or VP1) amino acid sequence corresponds to GenBank accession number ABZ10812.1 as accessed on 23 Sep. 2008 or SEQ ID NO: 202.

In some preferred embodiments, the acceptor AAV capsid serotype has a low seroprevalence and the donor AAV capsid serotype(s) has a higher seroprevalence than the acceptor AAV capsid serotype. Examples of acceptor AAV capsid serotype with a low seroprevalence include with no limitations: AAV8, AAV9, AAV5, AAV-LK03, AAVrh10, AAVrh74, AAV9.rh74, AAV9.rh74-P1. In some more preferred embodiments, the acceptor AAV capsid serotype is selected from the group consisting of: AAV8, AAV9, AAV5, AAV-LK03, AAVrh74, AAV9.rh74, AAV9.rh74-P1 and AAVrh10. In some preferred embodiments the donor AAV capsid serotype(s) is chosen from AAV13 and hybrid AAV2/13. In some more preferred embodiments, the donor AAV capsid serotype(s) is selected from the group consisting of AAV13, and the sequences SEQ ID NO: 2 to 30; preferably AAV13 and the sequences SEQ ID NO: 2 to 10, 18, 20-22, 29 and 30; still more preferably AAV13 and the sequences SEQ ID NO: 2, 10, 20, 21 and 30.

Step b) comprises the replacement of one to eleven (1, 2, 3, 4, 5, 6, 7, 8, 9, 10 or 11) HVR sequences of the acceptor capsid serotype chosen from HVR1, HVR2, HVR3, HVR4, HVR5, HVR6, HVR7, HVR8, HVR9, HVR10, and HVR12 with different HVR sequence(s) from the corresponding HVR(s) of the donor AAV capsid protein(s).

In some embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; preferably an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid). HVR11 sequence which is not replaced in the method according to the invention corresponds to the sequence from positions 621 to 687; preferably the sequence from positions 630 to 682; the indicated positions being determined by alignment with SEQ ID NO: 1 (VP1 of AAV8 or AAV8 capsid). These positions correspond to a large definition of the HVR sequences.

In some embodiments, step b) comprises replacing less than 8 HVR sequences of the acceptor AAV capsid protein with different HVR sequence(s) from the corresponding HVR(s) of the donor AAV capsid protein(s), e.g., the recombinant hybrid AAV capsid protein comprises less than 8 HVR sequences from the donor AAV capsid protein(s). In some preferred embodiments, step b) comprises replacing up to 6 HVR sequences; preferably up to 4 HVR sequences, of the acceptor AAV capsid protein with different HVR sequence(s) from the corresponding HVR(s) of the donor AAV capsid protein(s), e.g., the recombinant hybrid AAV capsid protein comprises up to 6 HVR sequences, preferably up to 4 HVR sequences from the donor AAV capsid protein(s). In some preferred embodiments the acceptor AAV capsid serotype is selected from the group consisting of: AAV8, AAV9, AAV5, AAV-LK03, AAVrh74, AAV9.rh74, AAV9.rh74-P1, and AAVrh10 and/or the donor AAV capsid serotype(s) is selected from the group consisting of AAV13, and the sequences SEQ ID NO: 2 to 30. In some preferred embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; preferably an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid). In some embodiments, step b) comprises replacing one or more or all of HVR5 to HVR10 sequences of the acceptor AAV capsid protein with different HVR sequence(s) from the corresponding HVR(s) of the donor AAV capsid protein(s), e.g., the recombinant hybrid AAV capsid protein comprises one or more or all of HVR5 to HVR10 sequences from the donor AAV capsid protein(s). In some preferred embodiments, step b) comprises replacing one or more or all of HVR5 to HVR8 sequences of the acceptor AAV capsid protein with different HVR sequence(s) from the corresponding HVR(s) of the donor AAV capsid protein(s), e.g., the recombinant hybrid AAV capsid protein comprises one or more or all of HVR5 to HVR8 sequences from the donor AAV capsid protein(s). In some more preferred embodiments, step b) comprises replacing at least HVR5 sequence of the acceptor AAV capsid protein with a different HVR5 sequence from the corresponding HVR of the donor AAV capsid protein(s). HVR5 may be replaced alone or with one or more or all of HVR6 to HVR10 of the acceptor AAV capsid protein. For example, step b) may comprise replacing HVR5, HVR5 to HVR8, HVR5 to HVR9 or HVR5 to HVR10. HVR5 is preferably replaced alone or with one or more or all of HVR6 to HVR8 of the acceptor AAV capsid protein. For example, step b) may comprise replacing HVR5 or HVR5 to HVR8. In some preferred embodiments the acceptor AAV capsid serotype is selected from the group consisting of: AAV8, AAV9, AAV5, AAV-LK03, AAVrh74, AAV9.rh74, AAV9.rh74-P1, and AAVrh10 and/or the donor AAV capsid serotype(s) is selected from the group consisting of AAV13, and the sequences SEQ ID NO: 2 to 30; preferably AAV13 and the sequences SEQ ID NO: 2 to 10, 18, 20-22, 29 and 30; still more preferably AAV13 and the sequences SEQ ID NO: 2, 10, 20, 21 and 30. In some preferred embodiments, the one or more HVR5 to HVR10 sequence(s) of the donor AAV capsid protein (replacement HVR sequences) and/or acceptor AAV capsid protein(s) (replaced HVR sequences) are selected from the group consisting of an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; and an HVR10 sequence from positions 576 to 613; more preferably an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; and an HVR10 sequence from positions 576 to 613; the indicated positions being determined by alignment with SEQ ID NO: 1 (VP1 of AAV8 or AAV8 capsid).

In some particular embodiments, step b) comprises replacing HVR5 to HVR8 sequences of the acceptor AAV capsid protein with HVR5 to HVR8 sequences of a donor AAV capsid serotype selected from AAV13 and any one of SEQ ID NO: 2 to 30; preferably AAV13, #704 (SEQ ID NO: 2); #1704 (SEQ ID NO: 10); #3086 (SEQ ID NO: 20); #1024 (SEQ ID NO: 22); #508 (SEQ ID NO: 9); #3142 (SEQ ID NO: 21); #2320 (SEQ ID NO: 29); #1010 (SEQ ID NO: 6); M258 (SEQ ID NO: 30); #1570 (SEQ ID NO: 18); #1602 (SEQ ID NO: 5); #667 (SEQ ID NO: 7); #129 (SEQ ID NO: 3); and #767 (SEQ ID NO: 8); still more preferably AAV13, #704 (SEQ ID NO: 2) and M258 (SEQ ID NO: 30); preferably wherein HVR5 sequence of the donor AAV capsid protein (replacement HVR5 sequence) and/or acceptor AAV capsid protein(s) (replaced HVR5 sequence) is from positions 446 to 485; HVR6 sequence is from positions 485 to 502; HVR7 sequence is from positions 499 to 516; and HVR8 sequence is from positions 509 to 531; more preferably wherein HVR5 is from positions 446 to 484; HVR6 sequence is from positions 490 to 500; HVR7 sequence is from positions 501 to 516; and HVR8 sequence is from positions 514 to 529; the indicated positions being determined by alignment with SEQ ID NO: 1 (VP1 of AAV8 or AAV8 capsid). In some preferred embodiments the acceptor AAV capsid serotype is selected from the group consisting of: AAV8, AAV9, AAV5, AAV-LK03, AAVrh74, AAV9.rh74, AAV9.rh74-P1 and AAVrh10.

In some embodiments, step b) comprises replacing any one of HVR1 to HVR10 and HVR12 of the acceptor AAV capsid protein with a different HVR sequence from the corresponding HVR of the donor AAV capsid protein, e.g., the recombinant hybrid AAV capsid protein comprises one HVR sequence from the donor AAV capsid protein. In some particular embodiments, step b) comprises replacing HVR5, HVR6, HVR7, or HVR8 of the acceptor AAV capsid with a different a different HVR sequence from the corresponding HVR from the donor AAV capsid protein, e.g., the recombinant hybrid AAV capsid protein comprises the HVR5, HVR6, HVR7 or HVR8 sequence from the donor AAV capsid protein. In some preferred embodiments, step b) comprises replacing any one of HVR1, HVR3, HVR5, HVR6, HVR7, HVR8, HVR9, HVR10 and HVR12; preferably one of HVR3, HVR5, HVR9, HVR10 or HVR12 of the acceptor AAV capsid with a different HVR sequence from the corresponding HVR from the donor AAV capsid protein. In some preferred embodiments, step b) comprises replacing HVR5, of the acceptor AAV capsid with a different a different HVR5 sequence from the donor AAV capsid protein, e.g., the recombinant hybrid AAV capsid protein comprises the HVR5 sequence from the donor AAV capsid protein. In some preferred embodiments the acceptor AAV capsid serotype is selected from the group consisting of: AAV8, AAV9, AAV-LK03, AAVrh74, AAV9.rh74, AAV9.rh74-P1, AAV5 and AAVrh10 and/or the donor AAV capsid serotype(s) is selected from the group consisting of AAV13, and the sequences SEQ ID NO: 2 to 30; preferably AAV13 and the sequences SEQ ID NO: 2 to 10, 18, 20-22, 29 and 30; still more preferably the sequences SEQ ID NO: 2, 10, 20, 21 and 30. In some other preferred embodiments, step b) comprises replacing any one of HVR1, HVR3, HVR6, HVR7, HVR8, HVR9, HVR10 and HVR12 of AAV8; preferably HVR3, HVR9, HVR10 or HVR12 of AAV8; with a different HVR sequence from the corresponding HVR from a donor AAV capsid protein selected from the group consisting of AAV13, and the sequences SEQ ID NO: 2 to 30; preferably SEQ ID NO: 2. In some preferred embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; still more preferably, an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid).

In some particular embodiments, HVR5 is from a donor AAV capsid serotype selected from the group consisting of: AAV13; #704 (SEQ ID NO: 2); #1704 (SEQ ID NO: 10); #3086 (SEQ ID NO: 20); #508 (SEQ ID NO: 9); #3142 (SEQ ID NO: 21); #M258 (SEQ ID NO: 30); #1570 (SEQ ID NO: 18); #2731 (SEQ ID NO:4); #1602 (SEQ ID NO: 5); #667 (SEQ ID NO: 7); #129 (SEQ ID NO: 3); and #767 (SEQ ID NO: 8); preferably HVR5 is from an AAV capsid serotype selected from the group consisting of the sequences SEQ ID NO: 2, 10, 20, 21 and 30. The HVR5 sequence is advantageously from positions 446 to 485; preferably from positions 446 to 484; the indicated positions being determined by alignment with SEQ ID NO: 1 (VP1 of AAV8 or AAV8 capsid). In some preferred embodiments, HVR5 comprises a sequence selected from the group consisting of SEQ ID NO: 175 to 186; preferably SEQ ID NO: 175 to 179. In some preferred embodiments, the acceptor AAV capsid serotype is selected from the group consisting of: AAV8, AAV9, AAV5, AAV-LK03, AAVrh74, AAV9.rh74, AAV9.rh74-P1, and AAVrh10.

In some preferred embodiments, the hybrid AAV capsid protein has an increased tropism for muscles and/or the central nervous system compared to the acceptor AAV capsid protein or the acceptor and donor AAV capsid proteins. In some particular embodiments, the hybrid AAV capsid protein has an increased tropism for kidney compared to the acceptor AAV capsid protein or the acceptor and donor AAV capsid proteins. In some particular embodiments, the hybrid AAV capsid protein has an increased tropism for heart and/or skeletal muscles compared to the acceptor AAV capsid protein or the acceptor and donor AAV capsid proteins. The hybrid AAV capsid protein has advantageously an increased tropism for different skeletal muscle groups; in particular the hybrid AAV capsid protein has an increased tropism for at least two skeletal muscle groups in mice selected from the group consisting of: Extensor Digitorum Longus (EDL), Soleus (Sol), Quadriceps (Qua), Triceps and Diaphragm or Soleus (Sol), Quadriceps (Qua), Triceps and Diaphragm. In some particular embodiments, the hybrid AAV capsid protein has a decreased tropism for an off-target tissue, advantageously the liver.

In some preferred embodiments, the hybrid AAV capsid protein has a seroprevalence equivalent to the seroprevalence the acceptor AAV capsid protein. In some more preferred embodiments, the hybrid AAV capsid protein has an increased tropism for muscles and/or the central nervous system compared to the acceptor AAV capsid protein or the acceptor and donor AAV capsid proteins and a seroprevalence which is equivalent to the seroprevalence of the acceptor AAV capsid protein.

In some preferred embodiments, the acceptor AAV capsid serotype has a low seroprevalence, the donor AAV capsid serotype has a higher seroprevalence than the acceptor and the hybrid AAV capsid protein has a seroprevalence equivalent to the seroprevalence of the acceptor AAV capsid protein. In some more preferred embodiments, the hybrid AAV capsid protein has an increased tropism in muscles and/or the central nervous system compared to the acceptor AAV capsid protein or the acceptor and donor AAV capsid proteins.

In some preferred embodiments, the acceptor AAV capsid protein is from an AAV serotype selected from the group consisting of: AAV8 and AAV9, still more preferably AAV8.

In some embodiments, the hybrid AAV capsid protein is an hybrid between two AAV capsid serotypes, preferably between an acceptor AAV capsid serotype having a low seroprevalence and a donor AAV capsid serotype having a higher seroprevalence than the acceptor AAV capsid serotype.

In some other embodiments, the hybrid AAV capsid protein is an hybrid between more than two AAV capsid serotypes, preferably between an acceptor AAV capsid serotype having a low seroprevalence and donor AAV capsid serotypes having a higher seroprevalence than the acceptor AAV capsid serotype.

In some embodiments, the method further comprises the step (c) of assaying the tropism of the hybrid AAV capsid protein obtained in step (b) by comparison with at least its parent acceptor capsid protein and (d) of selecting an hybrid AAV capsid protein having improved tropism compared to at least its parent acceptor capsid protein. In some preferred embodiments, the method further comprises the step (e) of assaying the seroprevalence of the hybrid AAV capsid protein and (f) selecting an hybrid AAV capsid protein having a seroprevalence equivalent to the seroprevalence of the acceptor AAV capsid.

In some embodiments, the method further comprises the step of inserting a cell-targeting peptide in the hybrid AAV capsid protein obtained in step (b), in particular a peptide known not to alter the seroprevalence of the capsid. In some particular embodiments, the cell-targeting peptide comprises the RGD motif. The incorporation of the RGD sequence into the viral capsid can target the vector to integrins, which are widely expressed on several cell types (Michelfelder S. et al. PLoS One. 2009; 4(4): e5122). In particular, the insertion of the peptide RGDLGLS in the HVR10 of AAV capsid leads to enhanced muscles targeting without any impact on capsid seroprevalence (WO 2019/193119). In some preferred embodiments, the peptide is of up to 30 amino acids and comprises or consists of any one of: RGDLGLS (SEQ ID NO: 167), LRGDGLS (SEQ ID NO: 168), LGRGDLS (SEQ ID NO: 169), LGLRGDS (SEQ ID NO: 170), LGLSRGD (SEQ ID NO: 171) and RGDMSRE (SEQ ID NO: 172); preferably SEQ ID NO: 167. The sequences comprising the RGD motif may be flanked by up to five or more amino acids at their N- and/or C-terminal ends, such as for example by GQSG (SEQ ID NO: 173) and AQAA (SEQ ID NO: 174), respectively at the N- and C-terminal end of the peptide. One or more peptide(s) comprising the RGD motif may be inserted into a site exposed on the AAV capsid surface. Sites on the AAV capsid which are exposed on the capsid surface and tolerate peptide insertions, i.e. do not affect assembly and packaging of the virus capsid, are well-known in the art and include for example the AAV capsid surface loops or antigenic loops (Girod et al., Nat. Med., 1999, 5, 1052-1056; Grifman et al., Molecular Therapy, 2001, 3, 964-975); other sites are disclosed in Rabinowitz et al., Virology, 1999, 265, 274-285; Wu et al., J. Virol., 2000, 74, 8635-8647. In some particular embodiments, the cell-targeting peptide is inserted in an HVR, in particular HVR3, HVR4, HVR5 or HVR10; preferably HVR10. In particular, the peptide(s) comprising the RGD motif are inserted around any of positions 261, 383, 449, 575 or 590 according to the numbering in SEQ ID NO: 162 (AAV9.rh74), preferably around position 449 or 590, more preferably around position 590. The positions are indicated by reference to SEQ ID NO: 255; one skilled in the art will be able to find easily the corresponding positions in another sequence after alignment with SEQ ID NO: 162. The insertion site is advantageously from positions 587 to 592 or 588 to 593 according to the numbering in SEQ ID NO: 162, preferably from positions 587 to 592. The insertion of the peptide may or may not cause the deletion of some or all of the residue(s) from the insertion site. The peptide advantageously replaces all the residues from positions 587 to 592 or 588 to 593 of the AAV capsid protein according to the numbering in SEQ ID NO: 162, preferably all of the residues from positions 587 to 592.

In some embodiments, the method is a high throughput method, wherein step (a) and step (b) are performed simultaneously to prepare different hybrid AAV capsid proteins, for example different hybrid AAV capsid proteins derived from the same acceptor and/or donor AAV capsid proteins. The high throughput method may comprise additional steps (c)-(d) and/or (e)-(f) as defined above.

### Hybrid AAV Capsids

The invention also relates to a recombinant hybrid AAV capsid protein with improved tissue tropism obtained or obtainable by the method of the present disclosure.

The recombinant hybrid AAV capsid protein may be derived from any different natural or artificial AAV serotypes used as acceptor and donor AAV capsid serotypes such as in particular those described in the present disclosure. The recombinant hybrid AAV capsid protein which is an hybrid between an acceptor AAV capsid serotype and donor AAV capsid serotype(s) comprises one to eleven (1, 2, 3, 4, 5, 6, 7, 8, 9, 10 or 11) HVR sequences from the donor AAV capsid protein(s) chosen from HVR1, HVR2, HVR3, HVR4, HVR5, HVR6, HVR7, HVR8, HVR9, HVR10, and HVR12 (replacement HVR sequence(s)) replacing the corresponding HVR sequence(s) of the acceptor capsid serotype (replaced HVR sequence(s)); the replacement HVR sequence(s)) have by definition an amino acid sequence which is different from that of the replaced HVR sequence(s).

In some embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; preferably, an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid).

In some preferred embodiments, the recombinant hybrid AAV capsid protein is an hybrid between an acceptor AAV capsid serotype having a low seroprevalence and a donor AAV capsid serotype(s) having a higher seroprevalence than the acceptor AAV capsid serotype. In some particular embodiments the acceptor AAV capsid serotype is selected from the group consisting of: AAV8, AAV9, AAV-LK03, AAVrh74, AAV9.rh74, AAV9.rh74-P1, AAV5 and AAVrh10, and/or the donor AAV capsid serotype(s) is selected from the group consisting of AAV13 and the sequences SEQ ID NO: 2 to 30; preferably AAV13 and the sequences SEQ ID NO: 2 to 10, 18, 20-22, 29 and 30; more preferably AAV13 and the sequences SEQ ID NO: 2, 10, 20, 21 and 30. In some preferred embodiments, the donor AAV capsid serotype is SEQ ID NO: 2.

In some embodiments, the recombinant hybrid AAV capsid protein comprises less than 8 HVR sequences from the donor AAV capsid protein(s). In some preferred embodiments, the recombinant hybrid AAV capsid protein comprises up to 6; preferably up to 4 HVR sequences from the donor AAV capsid protein(s).). In some preferred embodiments the acceptor AAV capsid serotype is selected from the group consisting of: AAV8, AAV9, AAV-LK03, AAVrh74, AAV9.rh74, AAV9.rh74-P1, AAV5 and AAVrh10 and/or the donor AAV capsid serotype(s) is selected from the group consisting of AAV13 and the sequences SEQ ID NO: 2 to 30; preferably AAV13 and the sequences SEQ ID NO: 2 to 10, 18, 20-22, 29 and 30; more preferably AAV13 and the sequences SEQ ID NO: 2, 10, 20, 21 and 30. In some preferred embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; preferably an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid).

In some embodiments, the recombinant hybrid AAV capsid protein comprises one or more of HVR5 to HVR10 sequences from the donor AAV capsid protein(s). In some preferred embodiments, the recombinant hybrid AAV capsid protein comprises one or more of HVR5 to HVR8 sequences from the donor AAV capsid protein(s). In some preferred embodiments, the recombinant hybrid AAV capsid protein comprises at least HVR5 sequence from the donor AAV capsid protein. The recombinant hybrid AAV capsid protein may comprise HVR5 alone or in combination with one or more or all of HVR6 to HVR10 from the donor capsid serotype; preferably HVR5 alone or in combination with one or more or all of HVR6 to HVR8 from the donor capsid serotype. In some preferred embodiments the acceptor AAV capsid serotype is selected from the group consisting of: AAV8, AAV9, AAV5, AAV-LK03, AAVrh74, AAV9.rh74, AAV9.rh74-P1 and AAVrh10 and/or the donor AAV capsid serotype(s) is selected from the group consisting of AAV13 and the sequences SEQ ID NO: 2 to 30; preferably AAV13 and the sequences SEQ ID NO: 2 to 10, 18, 20-22, 29 and 30; more preferably AAV13 and the sequences SEQ ID NO: 2, 10, 20, 21 and 30. In some preferred embodiments, the one or more HVR5 to HVR10 sequence(s) of the donor AAV capsid protein (replacement HVR sequences) and/or acceptor AAV capsid protein(s) (replaced HVR sequences) are selected from the group consisting of an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; more preferably an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; and an HVR10 sequence from positions 576 to 613; more preferably an HVR5 sequence from positions 446 to 484; an HVR6 sequence from positions 490 to 500; an HVR7 sequence from positions 501 to 516; and an HVR8 sequence from positions 514 to 529; the indicated positions being determined by alignment with SEQ ID NO: 1 (VP1 of AAV8 or AAV8 capsid). In some preferred embodiments, the recombinant hybrid AAV capsid protein comprises or consists of a sequence selected from the group consisting of the sequences SEQ ID NO: 33 to 36, 47 to 58 and 60 to 73; preferably SEQ ID NO: 35, 36, 47, 48, 50, 51, 58, 67 and 73; and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some particular embodiments, the recombinant hybrid AAV capsid protein comprises HVR5 to HVR8 sequences of an AAV serotype (donor AAV capsid serotype) selected from the group consisting of: AAV13, and any one of SEQ ID NO: 2 to 30; preferably AAV13, #704 (SEQ ID NO: 2); #1704 (SEQ ID NO: 10); #3086 (SEQ ID NO: 20); #1024 (SEQ ID NO: 22); #508 (SEQ ID NO: 9); #3142 (SEQ ID NO: 21); #2320 (SEQ ID NO: 29); #1010 (SEQ ID NO: 6); M258 (SEQ ID NO: 30); #1570 (SEQ ID NO: 18); #1602 (SEQ ID NO: 5); #667 (SEQ ID NO: 7); #129 (SEQ ID NO: 3); and #767 (SEQ ID NO: 8); still more preferably AAV13, #704 (SEQ ID NO: 2) and M258 (SEQ ID NO: 30). Preferably, wherein HVR5 sequence of the donor AAV capsid protein (replacement HVR5 sequence) and/or acceptor AAV capsid protein(s) (replaced HVR5 sequence) is from positions 446 to 485; HVR6 sequence is from positions 485 to 502; HVR7 sequence is from positions 499 to 516; and HVR8 sequence is from positions 509 to 531; still more preferably HVR5 sequence is from positions 446 to 484; HVR6 sequence is from positions 490 to 500; HVR7 sequence is from positions 501 to 512; and HVR8 sequence is from positions 514 to 529; the indicated positions being determined by alignment with SEQ ID NO: 1 (VP1 of AAV8 or AAV8 capsid). In some preferred embodiments the acceptor AAV capsid serotype is selected from the group consisting of: AAV8, AAV9, AAV-LK03, AAVrh74, AAV9.rh74, AAV9.rh74-P1, AAV5 and AAVrh10. In some preferred embodiments, the recombinant hybrid AAV capsid protein comprises or consists of a sequence selected from the group consisting of the sequences SEQ ID NO: 35, 58, 60 to 72; preferably SEQ ID NO: 35, 58, 67; and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some embodiments, the recombinant hybrid AAV capsid protein comprises one HVR sequence (HVR1, HVR2, HVR3, HVR4, HVR5, HVR6, HVR7, HVR8, HVR9, HVR10, or HVR12 from the donor AAV capsid protein. In some particular embodiments, the recombinant hybrid AAV capsid protein comprises one of the HVR5, HVR6, HVR7 or HVR8 sequence from the donor AAV capsid protein. In some preferred embodiments the recombinant hybrid AAV capsid protein comprises the HVR5 sequence from the donor AAV capsid protein. In some preferred embodiments the acceptor AAV capsid serotype is selected from the group consisting of: AAV8, AAV9, AAV5, AAV-LK03, AAVrh74, AAV9.rh74, AAV9.rh74-P1 and AAVrh10 and/or the donor AAV capsid serotype(s) is selected from the group consisting of AAV13 and the sequences SEQ ID NO: 2 to 30; preferably AAV13 and the sequences SEQ ID NO: 2 to 10, 18, 20-22, 29 and 30; still more preferably the sequences SEQ ID NO: 2, 10, 20, 21 and 30. In some other preferred embodiments, the recombinant hybrid AAV capsid protein is from AAV8 acceptor capsid and comprises one of the HVR1, HVR3, HVR6, HVR7, HVR8, HVR9, HVR10 or HVR12 sequence from a donor AAV capsid serotype selected from the group consisting of AAV13, and the sequences SEQ ID NO: 2 to 30; preferably SEQ ID NO: 2; still more preferably, the recombinant hybrid AAV capsid protein is from AAV8 acceptor capsid and comprises HVR3, HVR9, HVR10 or HVR12 sequence from a from a donor AAV capsid serotype selected from the group consisting of AAV13, and the sequences SEQ ID NO: 2 to 30; preferably SEQ ID NO: 2. In some preferred embodiments, the HVR sequence(s) of the donor AAV capsid protein (replacement HVR sequences) and acceptor AAV capsid protein(s) (replaced HVR sequences) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 485; an HVR6 sequence from positions 485 to 502; an HVR7 sequence from positions 499 to 516; an HVR8 sequence from positions 509 to 531; an HVR9 sequence from positions 531 to 570; an HVR10 sequence from positions 576 to 613; and an HVR12 sequence from positions 687 to 738; preferably an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1 (VP1 of AAV8 or AAV8 capsid). In some preferred embodiments, the recombinant hybrid AAV capsid protein comprises or consists of a sequence selected from the group consisting of the sequences SEQ ID NO: 36 to 43, 45, 47 to 57, 73; preferably SEQ ID NO: 36, 38, 42, 43, 45, 47, 48, 50, 51, 73, and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some particular embodiments HVR5 is from an AAV capsid serotype selected from the group consisting of: #704 (SEQ ID NO: 2); #1704 (SEQ ID NO: 10); #3086 (SEQ ID NO: 20); #508 (SEQ ID NO: 9); #3142 (SEQ ID NO: 21); #M258 (SEQ ID NO: 30); #1570 (SEQ ID NO: 18); #2731 (SEQ ID NO: 4); #1602 (SEQ ID NO: 5); #667 (SEQ ID NO: 7); #129 (SEQ ID NO: 3); and #767 (SEQ ID NO:8).

The HVR5 sequence of the donor AAV capsid protein (replacement HVR5 sequence) and/or acceptor AAV capsid protein(s) (replaced HVR5 sequence) is advantageously from positions 446 to 485; preferably from positions 446 to 484; the indicated positions being determined by alignment with SEQ ID NO: 1 (VP1 of AAV8 or AAV8 capsid). In some preferred embodiments, HVR5 comprises a sequence selected from the group consisting of SEQ ID NO: 175 to 186; preferably SEQ ID NO: 175 to 179. In some preferred embodiments the acceptor AAV capsid serotype is selected from the group consisting of: AAV8, AAV9, AAV5 AAV-LK03, AAVrh74, AAV9.rh74, AAV9.rh74-P1, and AAVrh10. In some preferred embodiments, the recombinant hybrid AAV capsid protein comprises or consists of a sequence selected from the group consisting of the sequences SEQ ID NO: 36, 47 to 57, 73; preferably SEQ ID NO: 36, 47, 48, 50, 51, 73; 3 and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some preferred embodiments, the acceptor AAV capsid protein is from an AAV serotype selected from the group consisting of: AAV8 and AAV9, still more preferably AAV8.

In some embodiments, the hybrid AAV capsid protein is an hybrid between two AAV capsid serotypes, preferably between an acceptor AAV capsid serotype having a low seroprevalence and a donor AAV capsid serotype having a higher seroprevalence than the acceptor AAV capsid serotype.

In some other embodiments, the hybrid AAV capsid protein is an hybrid between more than two AAV capsid serotypes, preferably between an acceptor AAV capsid serotype having a low seroprevalence and donor AAV capsid serotypes having a higher seroprevalence than the acceptor AAV capsid serotype.

In some preferred embodiments, the hybrid AAV capsid protein has an increased tropism for muscle and/or central nervous system compared to the acceptor AAV capsid protein or the acceptor and donor AAV capsid proteins. In some particular embodiments, the hybrid AAV capsid protein has an increased tropism for kidney compared to the acceptor AAV capsid protein or the acceptor and donor AAV capsid proteins. In some particular embodiments, the hybrid AAV capsid protein has an increased tropism for heart and/or skeletal muscles. The hybrid AAV capsid protein has advantageously an increased tropism for different skeletal muscle groups; in particular the hybrid AAV capsid protein has an increased tropism for at least two skeletal muscle groups in mice selected from the group consisting of: Extensor Digitorum Longus (EDL), Soleus (Sol), Quadriceps (Qua), Triceps and Diaphragm or Soleus (Sol), Quadriceps (Qua), Triceps and Diaphragm. In some particular embodiments, the hybrid AAV capsid protein has a decreased tropism for an off-target tissue, advantageously the liver. In particular embodiments, the hybrid AAV capsid protein having an increased tropism for muscle and/or central nervous system compared to the acceptor and donor AAV capsid proteins comprises or consists of a sequence selected from the group consisting of the sequences SEQ ID NO: 33 to 43, 45, 47 to 58, 60 to 73; preferably SEQ ID NO: 33 to 36, 38, 42, 43, 45, 47, 48, 50, 51, 58, 67, 73; 33 to 36 and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some preferred embodiments, the hybrid AAV capsid protein has a seroprevalence equivalent to the seroprevalence of the acceptor AAV capsid protein. In some more preferred embodiments, the hybrid AAV capsid protein is derived from an acceptor AAV capsid of low seroprevalence and a donor AAV capsid protein of a higher seroprevalence than the acceptor AAV capsid. In some more preferred embodiments, the hybrid AAV capsid protein has an increased tropism for muscle and/or central nervous system compared to the acceptor AAV capsid protein or the acceptor and donor AAV capsid proteins and a seroprevalence equivalent to the seroprevalence of the acceptor AAV capsid protein. In particular embodiments, the hybrid AAV capsid protein having an increased tropism for muscle and/or central nervous system compared to the acceptor AAV capsid protein or the acceptor and donor AAV capsid proteins and a seroprevalence equivalent to the seroprevalence of the acceptor AAV capsid protein comprises or consists of a sequence selected from the group consisting of the sequences SEQ ID NO: 35 to 43, 45, 47 to 58, 60 to 73; preferably SEQ ID NO: 35, 36, 38, 42, 43, 45, 47, 48, 50, 51, 58, 67, 73; and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

### Polynucleotide, Vector, and Use for AAV Vector Production

Another aspect of the invention is a polynucleotide encoding the recombinant hybrid AAV capsid protein in expressible form. The polynucleotide may be DNA, RNA or a synthetic or semi-synthetic nucleic acid.

In some embodiments, the polynucleotide encodes a recombinant hybrid AAV capsid protein having a sequence selected from the group consisting of the sequences SEQ ID NO: 33 to 43, 45, 47 to 58, 60 to 73; preferably SEQ ID NO: 35, 36, 38, 42, 43, 45, 47, 48, 50, 51, 58, 67, 73; and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In some preferred embodiments, the polynucleotide comprises or consists of a sequence selected from the group consisting of the sequences SEQ ID NO: 78, 80, 82, 84, 86, 88, 90, 92, 94, 96, 98, 102, 106, 108, 110, 112, 114, 116, 118, 120, 122, 124, 126, 128, 132, 134, 136, 138, 140, 142, 144, 146, 148, 150, 152, 154, 156, 158; preferably 82, 84, 88, 96, 98, 102, 106, 108, 112, 114, 128, 146, 158, and the sequences having at least 80%, 85%, 90%, 95%, 97%, 98% or 99% identity with said sequences. The polynucleotide is a functional polynucleotide sequence, which means that the sequence of the polynucleotide codes for the recombinant hybrid AAV capsid protein.

In some embodiments, the polynucleotide further encodes AAV Replicase (Rep) protein in expressible form, preferably Rep from AAV2.

The polynucleotide is advantageously inserted into a recombinant vector, which includes, in a non-limiting manner, linear or circular DNA or RNA molecules consisting of chromosomal, non-chromosomal, synthetic or semi-synthetic nucleic acids, such as in particular viral vectors, plasmid or RNA vectors. Numerous vectors into which a nucleic acid molecule of interest can be inserted in order to introduce it into and maintain it in a eukaryotic host cell are known per se; the choice of an appropriate vector depends on the use envisioned for this vector (for example, replication of the sequence of interest, expression of this sequence, maintaining of this sequence in extrachromosomal form, or else integration into the chromosomal material of the host), and also on the nature of the host cell.

In some embodiments, the vector is a plasmid.

The recombinant vector for use in the present invention is an expression vector comprising appropriate means for expression of the hybrid AAV capsid protein, and preferably also AAV Rep protein. Usually, each coding sequence (hybrid AAV Cap and AAV Rep) is inserted in a separate expression cassette either in the same vector or separately. Each expression cassette comprises the coding sequence (open reading frame or ORF) functionally linked to the regulatory sequences which allow the expression of the corresponding protein in AAV producer cells, such as in particular promoter, promoter/enhancer, intron, initiation codon (ATG), stop codon, transcription termination signal. Alternatively, the hybrid AAV Cap and the AAV Rep proteins may be expressed from a unique expression cassette using an Internal Ribosome Entry Site (IRES) inserted between the two coding sequences or a viral 2A peptide. In addition, the codon sequences encoding the hybrid AAV Cap, and AAV Rep if present, are advantageously optimized for expression in AAV producer cells, in particular human producer cells.

Another aspect of the invention is a cell stably transformed with a recombinant vector for expression of the hybrid AAV capsid protein, and preferably also AAV Rep protein. The cell stably expresses the hybrid AAV capsid and AAV Rep proteins (producer cell line). The producer cell is advantageously a human cell.

The vector, preferably a recombinant plasmid, and the producer cell line are useful for producing hybrid AAV vectors comprising the hybrid AAV capsid protein of the invention, using standard AAV production methods that are well-known in the art (Review in Aponte-Ubillus et al., Applied Microbiology and Biotechnology, 2018, 102: 1045-1054).

Briefly, following co-transfection of the producer cell line stably expressing the hybrid AAV capsid and AAV Rep proteins with plasmid containing recombinant AAV vector genome comprising the gene of interest inserted in an expression cassette, flanked by AAV ITRs, in the presence of sufficient helper function to permit packaging of the rAAV vector genome into AAV capsid particle, the cells are incubated for a time sufficient to allow the production of AAV vector particles, the cells are then harvested, lysed, and AAV vector particles are purified by standard purification methods such as affinity chromatography or Iodixanol or Cesium Chloride density gradient ultracentrifugation.

### AAV Particle, Cell

Another aspect of the invention is an AAV particle comprising the hybrid recombinant AAV capsid protein of the invention. Preferably, the AAV particle is a recombinant AAV (rAAV) vector particle, also named hybrid capsid serotype rAAV vector particle or hybrid serotype rAAV vector particle. The AAV vector particle is suitable for gene therapy directed to a target tissue or cells in the individual, in particular muscle, and/or CNS cells or tissue or other cells or tissues. The rAAV vector particle is packaging a gene of interest. The genome of the rAAV vector may either be a single-stranded or self-complementary double-stranded genome (McCarty et al, Gene Therapy, 2003, December, 10(26), 2112-2118). Self-complementary vectors are generated by deleting the terminal resolution site (trs) from one of the AAV terminal repeats. These modified vectors, whose replicating genome is half the length of the wild-type AAV genome have the tendency to package DNA dimers. The AAV genome is flanked by ITRs. In particular embodiments, the AAV vector is a pseudotyped vector, i.e. its genome and capsid are derived from AAVs of different serotypes. In some preferred embodiments, the genome of the pseudotyped vector is derived from AAV2. The rAAV vector particle may be obtained using the method of producing recombinant AAV vector particles of the invention.

By “gene of interest”, it is meant a gene useful for a particular application, such as with no limitation, diagnosis, reporting, modifying, therapy and genome editing.

For example, the gene of interest may be a therapeutic gene, a reporter gene or a genome-editing enzyme.

By “gene of interest for therapy”, “gene of therapeutic interest”, or “heterologous gene of interest”, it is meant a therapeutic gene or a gene encoding a therapeutic protein, peptide or RNA.

The gene of interest is any nucleic acid sequence capable of modifying a target gene or target cellular pathway, in cells of target organs, in particular muscle and/or CNS, or other target organs of interest. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a protein of interest for a particular application, (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA.

In some embodiments, the sequence of the gene of interest is optimized for expression in the treated individual, preferably a human individual. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites.

The gene of interest is a functional gene able to produce the encoded protein, peptide or RNA in the target cells of the disease, in particular muscle cells and/or cells of the CNS or other target cells of interest. In some embodiments, the gene of interest is a human gene. The AAV viral vector comprises the gene of interest in a form expressible in cells of target organs, in particular muscle cells, including cardiac and skeletal muscle cells muscles, and/or cells of the CNS or other target cell of interest. In particular, the gene of interest is operably linked to appropriate regulatory sequences for expression of a transgene in the individual's target cells, tissue(s) or organ(s). Such sequences which are well-known in the art include in particular a promoter, and further regulatory sequences capable of further controlling the expression of a transgene, such as without limitation, enhancer, terminator, intron, silencer, in particular tissue-specific silencer, and microRNA. The gene of interest is operably linked to a ubiquitous, tissue-specific or inducible promoter which is functional in cells of target organs, in particular muscle and/or CNS. The gene of interest may be inserted in an expression cassette further comprising additional regulatory sequences as disclosed above.

Examples of ubiquitous promoters include the CAG promoter, phosphoglycerate kinase 1 (PGK) promoter, the cytomegalovirus enhancer/promoter (CMV), the SV40 early promoter, the retroviral Rous sarcoma virus (RSV) LTR promoter, the dihydrofolate reductase promoter, the β-actin promoter, and the EF1 promoter. Muscle-specific promoters include without limitation, the desmin (Des) promoter, muscle creatine kinase (MCK) promoter, CK6 promoter, alpha-myosin heavy chain (alpha-MHC) promoter, myosin light chain 2 (MLC-2) promoter, cardiac troponin C (cTnC) promoter, synthetic muscle-specific SpC5-12 promoter, the human skeletal actin (HSA) promoter. Promoters for CNS expression include promoters driving ubiquitous expression and promoters driving expression into neurons. Representative promoters driving ubiquitous expression include, without limitation: CAG promoter (includes the cytomegalovirus enhancer/chicken beta actin promoter, the first exon and the first intron of the chicken beta-actin gene and the splice acceptor of the rabbit beta-globin gene); PGK (phosphoglycerate kinase 1) promoter; β-actin promoter; EF1a promoter; CMV promoter. Representative promoters driving expression into neurons include, without limitation, the promoter of the Calcitonin Gene-Related Peptide (CGRP), a known motor neuron-derived factor. Other neuron-selective promoters include the promoters of Choline Acetyl Transferase (ChAT), Neuron Specific Enolase (NSE), Synapsin, Hb9 and ubiquitous promoters including Neuron-Restrictive Silencer Elements (NRSE). Representative promoters driving selective expression in glial cells include the promoter of the Glial Fibrillary Acidic Protein gene (GFAP).

The RNA is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene involved in muscle disease. The guide RNA in complex with a Cas enzyme or similar enzyme for genome editing may be used to modify the sequence of a target gene, in particular to correct the sequence of a mutated/deficient gene or to modify the expression of a target gene involved in a disease, in particular a neuromuscular disease. The antisense RNA capable of exon skipping is used in particular to correct a reading frame and restore expression of a deficient gene having a disrupted reading frame. In some embodiments, the RNA is a therapeutic RNA.

The genome-editing enzyme according to the invention is any enzyme or enzyme complex capable of modifying a target gene or target cellular pathway, in particular in muscle cells. For example, the genome-editing enzyme may modify the expression, sequence or regulation of the target gene or cellular pathway. The genome-editing enzyme is advantageously an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double-strand break (DSB) or single-stranded DNA break (nickase such as Cas9(D10A) in the target genomic locus and is used for site-specific genome editing applications, including with no limitations: gene correction, gene replacement, gene knock-in, gene knock-out, mutagenesis, chromosome translocation, chromosome deletion, and the like. For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene causing a a muscle or central nervous system (CNS) disorder, such as for example a neuromuscular disease. Alternatively, the genome-editing enzyme, such as Cas enzyme and similar enzymes may be engineered to become nuclease-deficient and used as DNA-binding protein for various genome engineering applications such as with no limitation: transcriptional activation, transcriptional repression, epigenome modification, genome imaging, DNA or RNA pull-down and the like.

The invention also relates to an isolated cell, in particular a cell from an individual, which is stably transduced with a rAAV vector particle of the invention. The individual is advantageously a patient to be treated. In some embodiments, the cell is a muscle and/or CNS cell according to the present disclosure a progenitor of said cell or a pluripotent stem cell such as induced pluripotent stem cell (iPS cell), embryonic stem cells, fetal stem cell and adult stem cell.

### Pharmaceutical Composition and Therapeutic Uses

Another aspect of the invention is a pharmaceutical composition comprising at least an active agent selected from an AAV vector particle or a cell of the invention, and a pharmaceutically acceptable carrier.

The nucleic rAAV vector particle, cell and derived pharmaceutical composition of the invention may be used for treating diseases by gene therapy, in particular targeted gene therapy directed to muscle and/or CNS cells or tissue. The cell and derived pharmaceutical composition of the invention may be used for treating diseases by cell therapy, in particular cell therapy directed to muscle and/or CNS cell or other target cells of interest.

As used herein “Gene therapy” refers to a treatment of an individual which involves delivery of nucleic acid of interest into an individual's cells for the purpose of treating a disease. Delivery of the nucleic acid is generally achieved using a delivery vehicle, also known as a vector. The rAAV vector particle of the invention may be employed to deliver a gene to a patient's cells.

As used herein “Cell therapy” refers to a process wherein cells stably transduced by a rAAV vector particle of the invention are delivered to the individual in need thereof by any appropriate mean such as for example by intravenous injection (infusion), or injection in the tissue of interest (implantation or transplantation). In particular embodiments, cell therapy comprises collecting cells from the individual, transducing the individual's cells with the rAAV vector particle of the invention, and administering the stably transduced cells back to the patient. As used herein “cell” refers to isolated cell, natural or artificial cellular aggregate, bioartificial cellular scaffold and bioartificial organ or tissue.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed or an abnormal gene is suppressed (inactivated): this is gene editing therapy.

In additive gene therapy, the gene of interest may be a functional version of a gene, which is deficient or mutated in a patient, as is the case for example in a genetic disease. In such a case, the gene of interest will restore the expression of a functional gene. Thus, by gene editing or gene replacement a correct version of this gene is provided in target cells, in particular muscle and/or CNS cells or other target cells of affected patients, this may contribute to effective therapies against the disease.

Gene or genome editing uses one or more gene(s) of interest, such as:
a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

Gene therapy is used for treating various inherited (genetic) or acquired diseases or disorders affecting the structure or function of target tissue(s), in particular muscle(s) and/or the CNS, including skeletal or cardiac muscle(s), the brain or spinal cord. The diseases may be caused by trauma, infection, degeneration, structural or metabolic defects, tumors, autoimmune disorders, stroke or others. Non-limiting examples of diseases that can be treated by gene therapy include neuromuscular genetic disorders such as muscular genetic disorders; cancer; neurodegenerative diseases and auto-immune diseases.

In some embodiments, the target gene for gene therapy (additive gene therapy or gene editing) is a gene responsible for a neuromuscular disease. Neuromuscular genetic disorders include in particular: Muscular dystrophies, Congenital muscular dystrophies, Congenital myopathies, Distal myopathies, Other myopathies, Myotonic syndromes, Ion Channel muscle diseases, Malignant hyperthermia, Metabolic myopathies, Hereditary Cardiomyopathies, Congenital myasthenic syndromes, Motor Neuron diseases, Hereditary paraplegia, Hereditary motor and sensory neuropathies and other neuromuscular disorders. In some preferred embodiments, the target gene for gene therapy (additive gene therapy or gene editing) is a gene responsible for a neuromuscular disease selected from the group comprising Duchenne muscular dystrophy (DMD gene), Limb-girdle muscular dystrophies (LGMDs) (CAPN3, DYSF, FKRP, ANO5 genes and others), Spinal muscular atrophy (SMN1 gene), myotubular myopathy (MTM1 gene), Pompe disease (GAA gene) and Glycogen storage disease III (GSD3) (AGL gene).

Dystrophinopathies are a spectrum of X-linked muscle diseases caused by pathogenic variants in DMD gene, which encodes the protein dystrophin. Dystrophinopathies comprises Duchenne muscular dystrophy (DMD), Becker muscular dystrophy (BMD) and DMD-associated dilated cardiomyopathy.

The Limb-girdle muscular dystrophies (LGMDs) are a group of disorders that are clinically similar to DMD but occur in both sexes as a result of autosomal recessive and autosomal dominant inheritance. Limb-girdle dystrophies are caused by mutation of genes that encode sarcoglycans and other proteins associated with the muscle cell membrane, which interact with dystrophin. The term LGMD1 refers to genetic types showing dominant inheritance (autosomal dominant), whereas LGMD2 refers to types with autosomal recessive inheritance. Pathogenic variants at more than 50 loci have been reported (LGMD1A to LGMD1G; LGMD2A to LGMD2W). Calpainopathy (LGMD2A) is caused by mutation of the gene CAPN3 with more than 450 pathogenic variants described. Contributing genes to LGMD phenotype include: anoctamin 5 (ANO5), blood vessel epicardial substance (BVES), calpain 3 (CAPN3), caveolin 3 (CAV3), CDP-L-ribitol pyrophosphorylase A (CRPPA), dystroglycan 1 (DAG1), desmin (DES), DnaJ heat shock protein family (Hsp40) homolog, subfamily B, member 6 (DNAJB6), dysferlin (DYSF), fukutin related protein (FKRP), fukutin (Fla), GDP-mannose pyrophosphorylase B (GMPPB), heterogeneous nuclear ribonucleoprotein D like (HNRNPDL), LIM zinc finger domain containing 2 (LIMS2), lain A:C (LMNA), myotilin (MYOT), plectin (PLEC), protein O-glucosyltransferase 1 (PLOGLUT1), protein O-linked mannose N-acetylglucosaminyltransferase 1 (beta 1,2-) (POMGNT1), protein O-mannose kinase (POMK), protein O-mannosyltransferase 1 (POMT1), protein O-mannosyltransferase 2 (POMT2), sarcoglycan alpha (SGCA), sarcoglycan beta (SGCB), sarcoglycan delta (SGCD), sarcoglycan gamma (SGCG), titin-cap (TCAP), transportin 3 (TNPO3), torsin 1A interacting protein (TOR1AIP1), trafficking protein particle complex 11 (TRAPPC11), tripartite motif containing 32 (TRIM 32) and titin (TTN). Major contributing genes to LGMD phenotype include CAPN3, DYSF, FKRP and ANO5 (Babi Ramesh Reddy Nallamilli et al., Annals of Clinical and Translational Neurology, 2018, 5, 1574-1587.

Spinal muscular atrophy is a genetic disorder caused by mutations in the Survival Motor Neuron 1 (SMN1) gene which is characterized by weakness and wasting (atrophy) in muscles used for movement.

X-linked myotubular myopathy is a genetic disorder caused by mutations in the myotubularin (MTM1) gene which affects muscles used for movement (skeletal muscles) and occurs almost exclusively in males. This condition is characterized by muscle weakness (myopathy) and decreased muscle tone (hypotonia).

Pompe disease is a genetic disorder caused by mutations in the acid alpha-glucosidase (GAA) gene. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Glycogen storage disease III (GSD3) is an autosomal recessive metabolic disorder caused by homozygous or compound heterozygous mutation in the Amylo-Alpha-1, 6-Glucosidase, 4-Alpha-Glucanotransferase (AGL) gene which encodes the glycogen debrancher enzyme and associated with an accumulation of abnormal glycogen with short outer chains. Clinically, patients with GSD III present in infancy or early childhood with hepatomegaly, hypoglycemia, and growth retardation. Muscle weakness in those with Ma is minimal in childhood but can become more severe in adults; some patients develop cardiomyopathy.

Replacement or additive gene therapy may be used to treat cancer, in particular rhabdomyosarcomas. Genes of interest in cancer could regulate the cell cycle or the metabolism and migration of the tumor cells, or induce tumor cell death. For instance, inducible caspase-9 could be expressed in muscle cells to trigger cell death, preferably in combination therapy to elicit durable anti-tumor immune responses.

Gene editing may be used to modify gene expression in target cells, in particular muscle and/or CNS cells, in the case of auto-immunity or cancer, or to perturb the cycle of viruses in such cells. In such cases, preferably, the gene of interest is chosen from those encoding guide RNA (gRNA), site-specific endonucleases (TALEN, meganucleases, zinc finger nucleases, Cas nuclease), DNA templates and RNAi components, such as shRNA and microRNA. Tools such as CRISPR/Cas9 may be used for this purpose.

In some embodiments, gene therapy is used for treating diseases affecting other tissues, by expression of a therapeutic gene in target tissue, in particular, muscle and/or CNS tissue. This is useful to avoid expression of the therapeutic gene in the liver, in particular in patients having a concurrent hepatic disorder such as hepatitis. The therapeutic gene encodes preferably a therapeutic protein, peptide or antibody which is secreted from the muscle cells into the blood stream where it can be delivered to other target tissues such as for example the liver. Examples of therapeutic genes include with no limitation: Factor VIII, Factor IX and GAA genes.

In the various embodiments of the present invention, the pharmaceutical composition comprises a therapeutically effective amount of rAAV vector particle or cell. In the context of the invention a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The invention provides also a method for treating a disease by expression of a therapeutic gene in a target tissue, in particular muscle and/or CNS tissue, comprising: administering to a patient a therapeutically effective amount of the pharmaceutical composition as described above.

Another aspect of the invention relates to the rAAV vector particle, cell, pharmaceutical composition according to the present disclosure as a medicament, in particular for use in the treatment of a muscle or CNS disorder according to the present disclosure, in particular neuromuscular genetic disease.

The invention provides also a method for treating a muscle or CNS disorder, comprising: administering to a patient a therapeutically effective amount of the pharmaceutical composition as described above, comprising at least an active agent selected from an AAV vector particle or a cell of the invention, and a pharmaceutically acceptable carrier.

A further aspect of the invention relates to the use of a rAAV vector particle, cell according to the present disclosure in the manufacture of a medicament for the treatment of a muscle or CNS disorder, in particular neuromuscular genetic disease.

Another aspect of the invention relates to the use of a rAAV vector particle or a cell of the present disclosure for the treatment of a muscle or CNS disorder according to the present disclosure, in particular neuromuscular genetic disease.

A further aspect of the invention relates to a pharmaceutical composition for treatment of a muscle or CNS disorder according to the present disclosure, in particular neuromuscular genetic disease, comprising an AAV vector particle or a cell of the present disclosure as an active component.

A further aspect of the invention relates to a pharmaceutical comprising an AAV vector particle or a cell of the present disclosure for treating a muscle or CNS disorder according to the present disclosure, in particular neuromuscular genetic disease,

As used herein, the term “patient” or “individual” includes human and other mammalian subjects that receive either prophylactic or therapeutic treatment. Preferably, a patient or individual according to the invention is a human.

Treatment”, or “treating” as used herein, is defined as the application or administration of a therapeutic agent or combination of therapeutic agents to a patient, or application or administration of said therapeutic agents to an isolated tissue or cell line from a patient, who has a disease, in particular a muscle or CNS disorder with the purpose to cure, heal, alleviate, relieve, alter, remedy, ameliorate, improve or affect the disease, or any symptom of the disease. In particular, the terms “treat’ or treatment” refers to reducing or alleviating at least one adverse clinical symptom associated with the disease.

The term “treatment” or “treating” is also used herein in the context of administering the therapeutic agents prophylactically.

The pharmaceutical composition of the present invention is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). The administration can be systemic, local or systemic combined with local; systemic includes parenteral and oral, and local includes local and loco-regional. Systemic administration is preferably parenteral such as subcutaneous (SC), intramuscular (IM), intravascular such as intravenous (IV) or intraarterial; intraperitoneal (IP); intradermal (ID), epidural or else. The parenteral administration is advantageously by injection or perfusion. Local administration is preferably intracerebral, intracerebroventricular, intracisternal, and/or intrathecal administration. The administration may be for example by injection or perfusion. In some preferred embodiments, the administration is parenteral, preferably intravascular such as intravenous (IV) or intraarterial. In some other preferred embodiments, the administration is intracerebral, intracerebroventricular, intracisternal, and/or intrathecal administration, alone or combined with parenteral administration, preferably intravascular administration. In some other preferred embodiments, the administration is parenteral, preferably intravascular alone or combined with intracerebral, intracerebroventricular, intracisternal, and/or intrathecal administration.

The various embodiments of the present disclosure can be combined with each other and the present disclosure encompasses the various combinations of embodiments of the present disclosure.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1. Representation of AAV hybrids and parental capsids

On the top, schematic representation of VP1 amino acid sequence of AAV8 with the localization of the 12 HVRs (black boxes). The number of each HVR is indicated on the corresponding box. The amino acid coordinates indicate the position used for HVR substitutions. On the bottom, schematic representation of AAV8, #704 and the 6 hybrids capsids. VP1 amino acid sequences were multialigned using the ClustalW algorithm and compared to AAV8 sequences (in black). Grey colour indicates the amino acid variations specific to #704 capsid and present in the mutants.

FIG. 2. Specific tissue targeting of hybrid capsids

Luciferase activity of controls and new hydrid capsids. Each column represents the average of activity in at least 3 mice expressed as fold change versus AAV8. Standard deviations are displayed. Statistical analysis on fold change was performed using one-way ANOVA. Dunnett's multiple comparison test was used to compare the mean of each capsid with the mean of the controls (* versus #704, #versus AAV8). *, #=p<0.05; **, ##=p<0.01; ***, ###=p<0.001.

FIG. 3. Seroprevalence of new hybrid capsids

Level of anti-AAV capsid antibodies in hybrid and parental capsids assessed by ELISA in a cohort of 46 human sera. For each capsid, the sera are categorized in 3 groups according to the level of anti-AAV IgG. Statistical analysis is performed using χ² test with Monte Carlo simulation.

FIG. 4. Presence of anti-AAV capsid antibodies against new hybrid capsids

Presence of anti-AAV capsid antibodies assessed by ELISA in a pool of human IVIg. The level of antibodies in parental capsids is compared to A) mutant 1, B) mutant 2, C) mutant 3, D) mutant 4 and E) mutant 5. X-axis represents the serial dilutions of IVIg, y-axis the normalized OD values related to the presence of anti-AAV antibodies. The OD50 of each capsid is displayed in the graphs. F) OD50 of parental and hybrid capsids resulted from 2 independent experiments. Standard deviations are displayed. Statistical analysis was performed using one-way ANOVA. Dunnett's multiple comparison test was used to compare the mean of each capsid with the mean of the controls (* versus AAV8, #versus #704). *, #=p<0.05; **, ##=p<0.01; ***, ###=p<0.001.

FIG. 5. Specific tissue targeting of mutants with single HVR substitution

Luciferase activity of AAV8 and new hydrid capsids in six different organs. Each column represents the average of activity in at least 3 mice expressed as fold change versus AAV8. Standard deviations are displayed. Statistical analysis on fold change was performed using one-way ANOVA. Dunnett's multiple comparison test was used to compare the mean of each capsid with the mean of AAV8. *=p<0.05; **=p<0.01; ***=p<0.001.

FIG. 6. Anti-AAV capsid antibodies in mutants with single HVR substitutions

Presence of anti-AAV capsid antibodies assessed by ELISA in a pool of human IVIg. The level of antibodies in parental capsids is compared to A) AAV8-mut.HVR1, B) AAV8-mut.HVR3, C) AAV8-mut.HVR6, D) AAV8-mut.HVR7, E) AAV8-mut.HVR8, F) AAV8-mut.HVR9, G) AAV8-mut.HVR10, H) AAV8-mut.HVR11 and I) AAV8-mut.HVR12. X-axis represents the dilutions of IVIg, y-axis the normalized OD values related to the presence of anti-AAV antibodies. The OD50 of each capsid is displayed in the graphs. J) OD50 of parental and hybrid capsids resulted from 2 independent experiments. Standard deviations are displayed. Statistical analysis was performed using one-way ANOVA. Dunnett's multiple comparison test was used to compare the mean of each capsid with the mean of the controls (* versus AAV8, #versus #704). *, #=p<0.05; **, ##=p<0.01; ***, ###=p<0.001.

FIG. 7. Specific tissue targeting of mutants with different HVR5 substitutions

Luciferase activity of AAV8 and new hydrid capsids in six different organs. Each column represents the average of activity in at least 3 mice expressed as fold change versus AAV8. Standard deviations are displayed. Statistical analysis on fold change was performed using one-way ANOVA. Dunnett's multiple comparison test was used to compare the mean of each capsid with the mean of AAV8. *=p<0.05; **=p<0.01; ***=p<0.001.

FIG. 8. Specific tissue targeting of mutants with different HVR5-8 combinations

Luciferase activity of AAV8 and new hydrid capsids in five different organs. Each column represents the average of activity in at least 3 mice expressed as fold change versus AAV8. Standard deviations are displayed. Statistical analysis on fold change was performed using one-way ANOVA. Dunnett's multiple comparison test was used to compare the mean of each capsid with the mean of AAV8. *=p<0.05; **=p<0.01; ***=p<0.001.

FIG. 9. Specific tissue targeting of AAV9 mutants

Luciferase activity of AAV9 and AAV9-R5-704 capsids in six different organs. Each column represents the average of activity in at least 3 mice expressed as fold change versus AAV9. Standard deviations are displayed. Statistical analysis on fold change was performed using one-way ANOVA. Dunnett's multiple comparison test was used to compare the mean of each capsid with the mean of PBS. *=p<0.05; **=p<0.01; ***=p<0.001.

FIG. 10. Level of anti-AAV capsid antibodies in AAV9 hybrid capsids

Presence of anti-AAV capsid antibodies assessed by ELISA in a pool of human IVIg. A) The level of antibodies of parental capsids is compared to AAV9-R5-704. X-axis represents the serial dilutions of IVIg, y-axis the normalized OD values related to the presence of anti-AAV antibodies. The OD50 of each capsid is displayed in the graph. B) OD50 of parental and hybrid capsids resulted from 2 independent experiments. Standard deviations are displayed. Statistical analysis was performed using one-way ANOVA. Dunnett's multiple comparison test was used to compare the mean of each capsid with the mean of the controls (* versus AAV8, #versus #704). *, #=p<0.05; **, ##=p<0.01; ***, ###=p<0.001.

### EXAMPLES

### Materials and Methods

### 1. Plasmid Construction for New Hybrid Capsids

To construct the plasmid containing AAV2 Rep sequence and the new hybrid Cap genes, the capsid sequences were synthesized (GENEWIZ). The fragment was inserted in the plasmid pAAV2 which contains AAV2 Rep and AAV2 Cap in order to replace the AAV2 Cap with the corresponding new Cap sequence.

### 2. AAV Production

HEK293T cells were grown in suspension in 50 mL of serum-free medium. The cells were transfected with 3 plasmids: i) a transgene plasmid, containing AAV2 ITRs flanking an expression cassette ii) the helper plasmid pXX6, containing adenoviral sequences necessary for AAV production, and iii) a plasmid containing AAV Rep and Cap genes, defining the serotype of AAV. Two days after transfection, the cells were lysed to release the AAV particles.

The viral lysate was purified by affinity chromatography. Viral genomes were quantified by a TaqMan real-time PCR assay using primers and probes corresponding to the ITRs of the AAV vector genome (Rohr et al. J Virol Methods., 2002, 106, 81-8.doi: 10.1016/s0166-0934(02)00138-6).

### 3. In Vivo Studies

All mouse studies were performed according to the French and European legislation on animal care and experimentation (2010/63/EU) and approved by the local institutional ethical committee (protocol no. 2016-002C). AAV vectors were administered intravenously via the tail vein to 6 weeks old male C57Bl6/J mice. PBS-injected littermates were used as controls. 15 days after vector injections, tissues were harvested and homogenized in DNAse/RNAse free water using Fastprep tubes (6.5 m/s; 60 secondes).

### 4. Luciferase Activity

Luciferase assay was used to measure the expression of the reporter gene used as transgene. Tissue lysates were centrifuged at 10000 rpm for 10 min, the supernatant was diluted in lysis buffer in a white opaque 96-well plate. Luciferase activity was measured using EnSpire (PerkinElmer) through sequential injections of assay buffer containing ATP and luciferine.

Protein quantification was performed on the samples using BCA assay in order to normalize the RLU (relative luminescence unit) on the quantity protein. The final results were expressed as RLU/mg of protein and normalized as fold change versus AAV8 control.

### 5. Seroprevalence of the Capsids

ELISA was performed to assess the presence of anti-AAV capsid antibodies (Ab) in a cohort of human sera and in a commercial pool of human intravenous immunoglobulin (IVIg), prepared from the serum of 1000-1500 donors per batch. AAV capsids were coated at 1×10E9 vg/well on Maxisorp™ plates (Nunc) and incubated overnight at 4° C. Plates were washed three times with PBS containing 6% milk and incubated at room temperature for two hours. Plates were washed three times with PBS containing 0.05% Tween (PBS-T) and incubated one hour at 37° C. with the sera dilutions. Each sera sample was analyzed using 4 logarithmic serial dilutions (from 1:10 to 1:10000), whereas the pool of IVIg was analyzed using 8 semi-log serial dilutions (from 1:10 to 1:316000). Plates were washed three times with PBS-T and incubated one hour at 37° C. with a goat anti-human IgG conjugated with HRP (1:10000 dilution). Plates were washed three times with PBS-T and added with TMB substrate. The reaction was stopped with H2SO4 and the optical density (OD of the plates was read at 492 nm. For the analysis of human sera, a standard curve of IVIg was used to determine the level of anti-AAV capsid IgG in each tested serum. Results are expressed in μg of anti-AAV capsid IgG per ml of serum. Sera with an ELISA IgG titer less than 10 μg/ml were considered as seronegative. For the analysis of IVIg samples, the OD values of each capsid were expressed as percentage of signal and analyzed on Prism. A model of dose-response curve was used to determine the IVIg dilution at which a reduction of 50% of the OD signal was observed (OD50). The OD50 of the hybrid capsids were compared to those of the parental capsids.

### Example 1: Production and In Vivo Testing of Hybrid Capsid from AAV8 with Hypervariable Regions from Other AAV Serotypes

The design of capsids hereby described is based on the combination of the hypervariable regions (HVR) of two selected parental capsids: the well-known AAV8 serotype and the newly isolated AAV2/13 sequence. The aim of the rational shuffling strategy is to transfer capsid properties from donor to acceptor capsid without alteration of acceptor capsid seroprevalence. VP1 sequence from AAV2/13 was obtained by aligning all AAV2/13 sequences isolated from human liver (La Bella T et al., Gut., 2020, 69, 737-747.doi: 10.1136/gutjnl-2019-318281;), the resulting amino acid consensus sequence is equal to the sequence #704 isolated in human. The consensus AAV2/13 sequence will hereinafter be called #704 (SEQ ID NO: 2). AAV8 capsid corresponds to SEQ ID NO: 1.

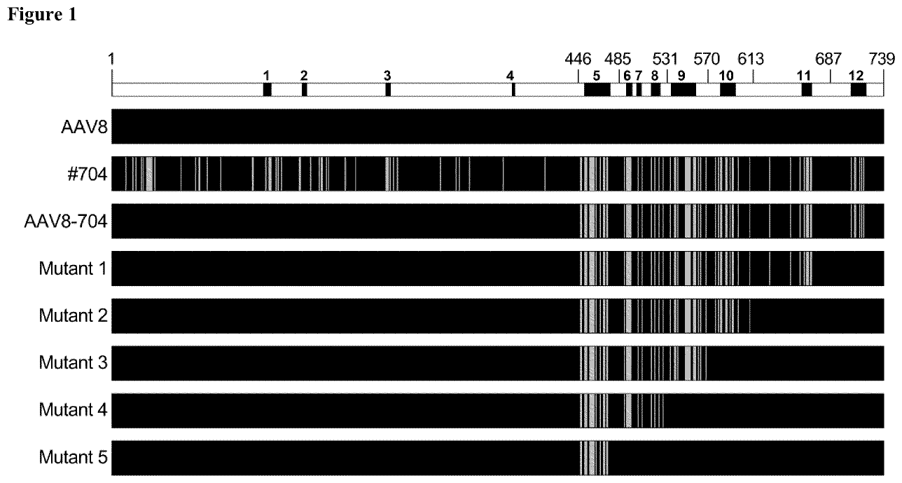

The inventors have developed 6 hybrid capsids corresponding to a variable number of HVRs (FIG. 1):
AAV8-704 composed by AAV8 from amino acid 1 to 446 and #704 from aa 447 to 739. This hybrid includes the HVRs 1 to 4 of AAV8 and 5 to 12 of #704. AAV8-704 has the amino acid sequence SEQ ID NO: 31 and is encoded by the polynucleotide of SEQ ID NO: 74.
Mutant 1 composed by AAV8 from amino acid 1 to 446, #704 from aa 447 to 687 and AAV8 from 688 to 739. This hybrid includes the HVRs 1, 2, 3, 4 and 12 of AAV8 and 5 to 11 of #704. Mutant 1 has the amino acid sequence SEQ ID NO: 32 and is encoded by the polynucleotide of SEQ ID NO: 76.
Mutant 2 composed by AAV8 from amino acid 1 to 446, #704 from aa 447 to 613 and AAV8 from 614 to 739. This hybrid includes the HVRs 1, 2, 3, 4, 11 and 12 of AAV8 and 5 to 10 of #704. Mutant 2 has the amino acid sequence SEQ ID NO: 33 and is encoded by the polynucleotide of SEQ ID NO: 78.
Mutant 3 composed by AAV8 from amino acid 1 to 446, #704 from aa 447 to 570 and AAV8 from 571 to 739. This hybrid includes the HVRs 1, 2, 3, 4, 10, 11 and 12 of AAV8 and 5 to 9 of #704. Mutant 3 has the amino acid sequence SEQ ID NO: 34 and is encoded by the polynucleotide of SEQ ID NO: 80.
Mutant 4 composed by AAV8 from amino acid 1 to 446, #704 from aa 447 to 531 and AAV8 from 532 to 739. This hybrid includes the HVRs 1, 2, 3, 4, 9, 10, 11 and 12 of AAV8 and 5 to 8 of #704. Mutant 4 has the amino acid sequence SEQ ID NO: 35 and is encoded by the polynucleotide of SEQ ID NO: 82.
Mutant 5 composed by AAV8 from amino acid 1 to 446, #704 from aa 447 to 485 and AAV8 from 486 to 739. This hybrid includes all HVRs of AAV8 except the HVR5 from #704. Mutant 5 has the amino acid sequence SEQ ID NO: 36 and is encoded by the polynucleotide of SEQ ID NO: 84.

### Capsids Production, In Vivo Biodistribution and Seroprevalence

Recombinant AAV vectors were produced by cloning the mutated Cap genes described above in a plasmid suitable for AAV vector production. A transgene expression cassette flanked by AAV2 ITRs and expressing a luciferase reporter gene was encapsidated in the so derived AAV vectors. Triple transfection of HEK293 cells was used to produce the vectors followed by immunoaffinity column purification. All capsid sequences were efficiently produced as AAV vectors, except AAV8-704 which was excluded from the following in vivo analysis.

TABLE 1
Production of AAV vectors with hybrid capsid serotype

| Capsids | Median (vg/ml) |
|---|---|
| AAV8 | 1.1E+11 |
| #704 | 6.6E+10 |
| AAV8-704 | 3.3E+10 |
| Mutant 1 | 1.4E+11 |
| Mutant 2 | 4.9E+11 |
| Mutant 3 | 4.6E+11 |
| Mutant 4 | 1.6E+11 |
| Mutant 5 | 3.6E+11 |

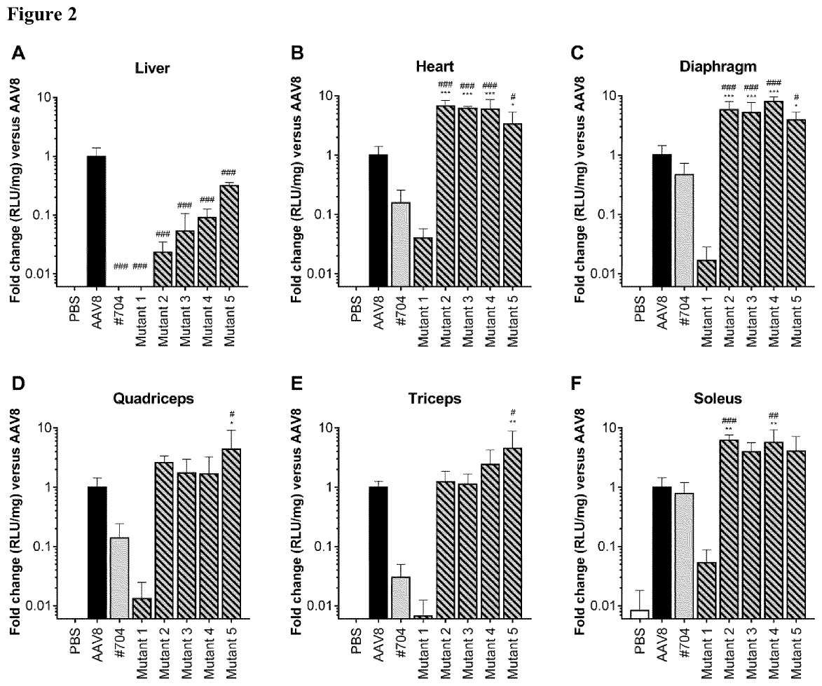

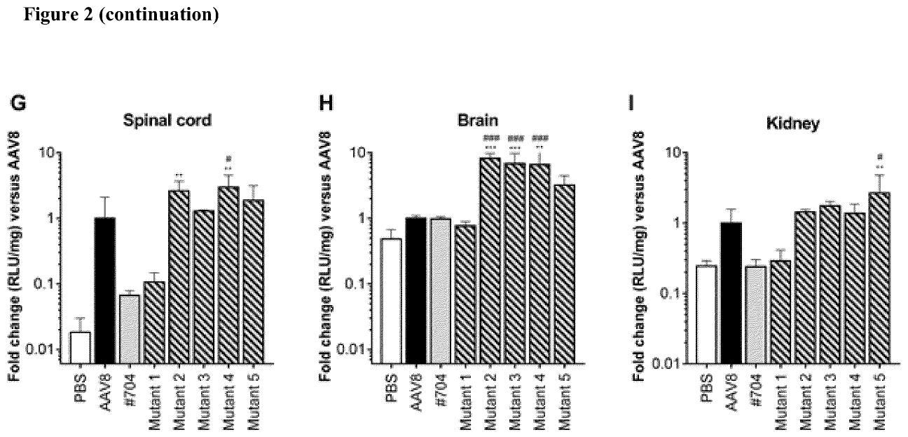

The vectors were tested in wild-type C57Bl6/J mice through intravenous injection of the different vectors at the dose of 1×10¹¹ vg/mice. Fifteen days post-injection, animals were sacrificed and the levels of expression of the transgene were measured in isolated tissues (liver, spleen, quadriceps, triceps, diaphragm, heart, kidneys, brain, soleus, spinal-cord). Results were expressed as RLU (relative luminescence unit) per mg of protein and normalized as fold change versus AAV8 control (Table 2 and FIG. 2).

TABLE 2
Transgene expression in tissues (Fold change (RLU/mg) versus AAV8

| | | | | | | | Spinal | | | |
|---|---|---|---|---|---|---|---|---|---|---|
| | Liver | Heart | Diaphragm | Quadriceps | Triceps | Soleus | cord | Brain | Kidney | Spleen |
| PBS | 0.0 | 0.0 | 0.0 | 0.0 | 0.0 | 0.0 | 0.0 | 0.5 | 0.2 | 0.4 |
| AAV9 | 0.3 | 1.9 | 0.6 | 0.5 | 0.4 | 0.5 | 0.4 | 1.7 | 1.0 | 0.4 |
| AAV8 | 1.0 | 1.0 | 1.0 | 1.0 | 1.0 | 1.0 | 1.0 | 1.0 | 1.0 | 1.0 |
| #704 | 0.0 | 0.2 | 0.5 | 0.1 | 0.0 | 0.8 | 0.1 | 1.0 | 0.2 | 0.5 |
| Mutant 1 | 0.0 | 0.0 | 0.0 | 0.0 | 0.0 | 0.1 | 0.1 | 0.8 | 0.3 | 0.2 |
| Mutant 2 | 0.0 | 6.7 | 5.8 | 2.6 | 1.2 | 6.3 | 2.6 | 8.3 | 1.4 | 0.4 |
| Mutant 3 | 0.1 | 6.1 | 5.2 | 1.7 | 1.1 | 4.0 | 1.3 | 6.8 | 1.8 | 0.3 |
| Mutant 4 | 0.1 | 6.0 | 8.0 | 1.7 | 2.4 | 5.7 | 3.0 | 6.6 | 1.4 | 0.8 |
| Mutant 5 | 0.3 | 3.3 | 3.9 | 4.4 | 4.5 | 4.1 | 1.9 | 3.2 | 2.7 | 1.2 |

In the liver (FIG. 2A), the luciferase activity of AAV8 was significantly higher than all the other tested capsids. The parental capsid #704 completely detargeted the liver. Luciferase activity in liver of mice injected with mutant capsids progressively increased in capsids containing more HVRs from AAV8 reaching the highest level in mutant 5 bearing HVR5 of capsid #704.

In all tested muscles (FIG. 2B-F), all mutant capsids except mutant 1 outperformed the parental capsid #704 and showed higher efficiency to AAV8.

Increased transduction levels were also observed in the spinal cord for all mutants, in particular mutants 2 and 4, compared to the parental capsids (FIG. 2G), whereas a very low luciferase activity was observed for #704 and mutant 1.

In the brain (FIG. 2H), the luciferase activity of AAV8, #704 and mutant 1 was comparable to PBS injected mice. Interestingly, mutants 2, 3, 4 and 5 were able to target the brain with higher efficiency than AAV9, AAV8 and #704 (Table 2).

Finally, in contrast to #704 and mutant 1, mutant 5 was able to target the kidney with higher luciferase expression than AAV8. (FIG. 2I).

Taken together, these results showed that the novel AAV mutated capsids exhibit an increased tropism for muscles and CNS compared to their parental capsids suggesting that the combination of hypervariable regions from AAV8 and wild type #704 may represent a promising strategy for the development of novel capsids.

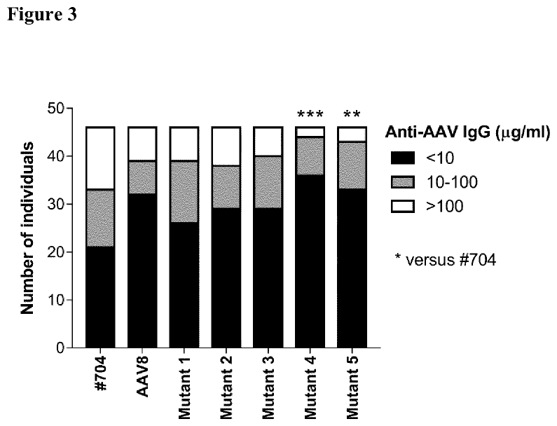

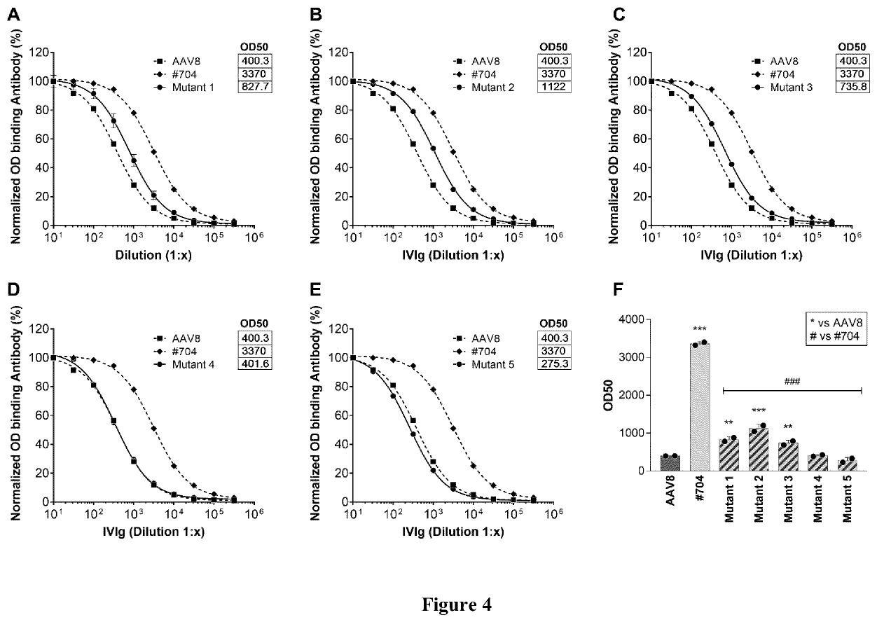

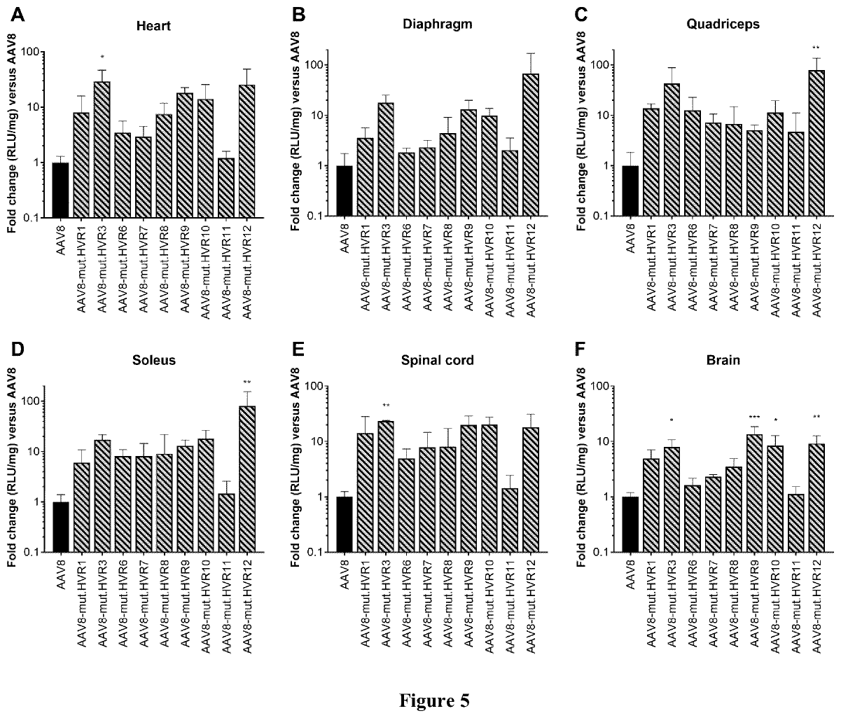

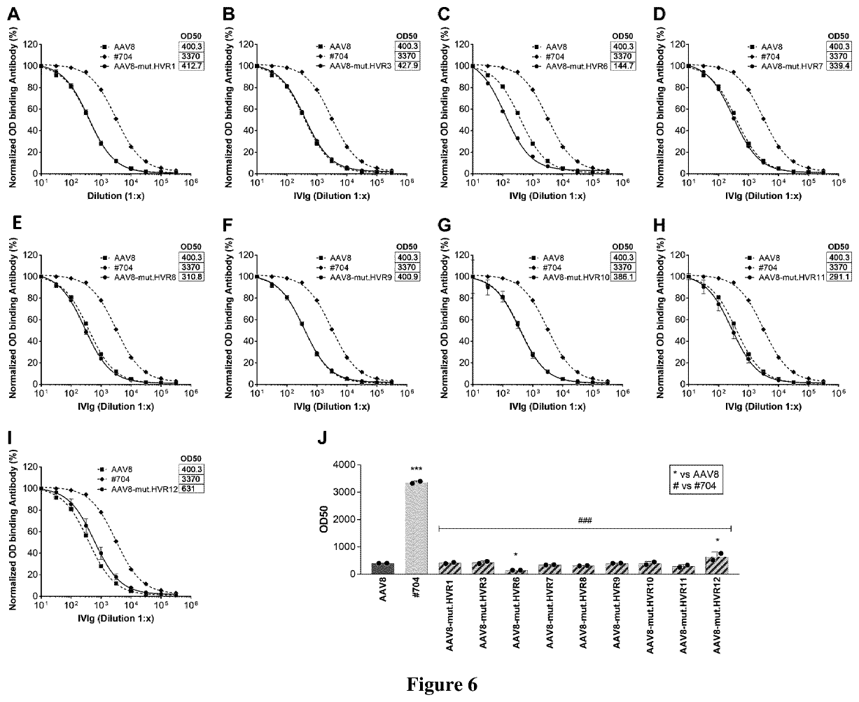

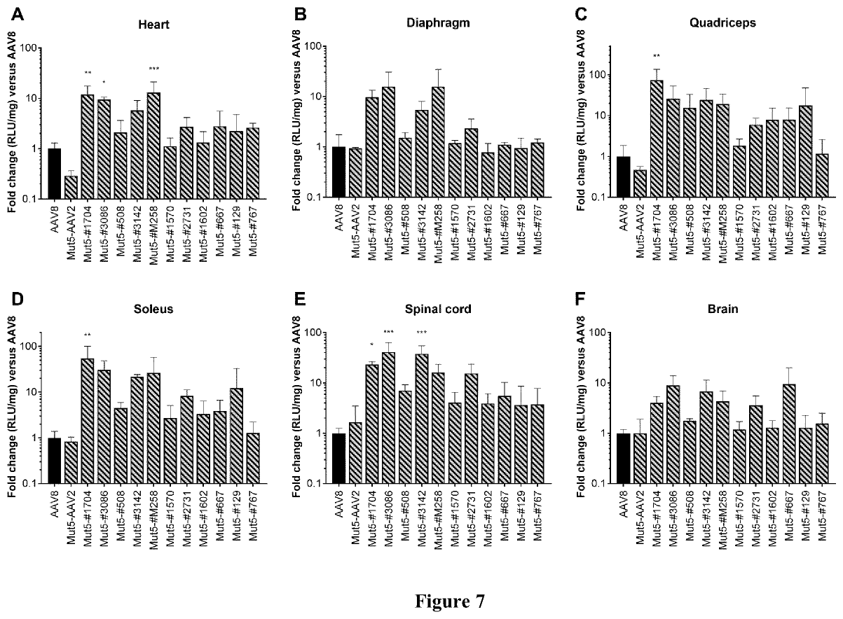

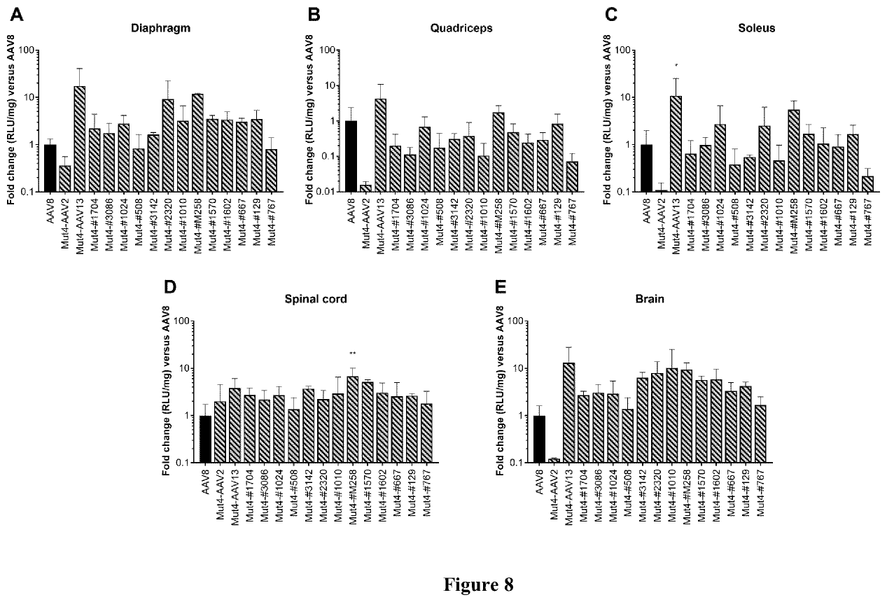

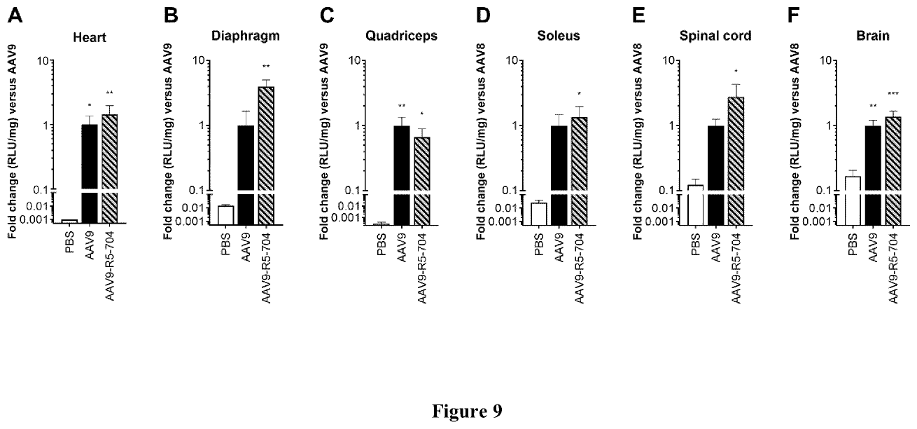

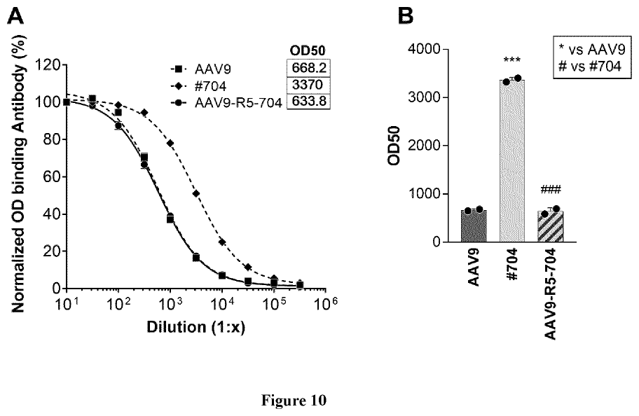

The inventors aimed at the identification of the minimal number of HVR regions that can be modified in a capsid without affecting capsid seroprevalence. The seroprevalence of the hybrid capsids was tested in parallel with the 2 parental capsids, AAV8 and #704. ELISA was performed to assess the presence of anti-AAV capsid antibodies (Ab) in a cohort of 46 human sera. As expected, giving the human origin of this capsid, the number of seropositive individuals was the highest for wild type #704 (n=25; FIG. 3). Decreasing number of seropositive individuals were observed for all mutants. In particular, mutant 4 and 5 showed significantly lower seropositive samples (n=10 and n=13, respectively) than parental capsid #704. Globally, the frequency of seropositive individuals gradually decreased in hybrid capsids containing lower number of HVR of #704 than AAV8 reaching 22% and 28% in mutant 4 and 5, respectively. Considering that the parental AAV8 capsid showed 30% of seropositivity in the tested cohort, these data suggest that the modifications of the HVR5, 6, 7 and 8 do not modify the immunogenic profile of the capsids. Similar results were also confirmed by analysing the level of anti-AAV capsid antibodies in a pool of human IVIg (FIG. 4). The OD50, defined as the IVIg dilution at which a reduction of 50% of the OD signal is observed, was used to compare the level of anti-AAV antibodies against the hybrids and parental capsids. An OD50 of 400 and 3370 was obtained for AAV8 and #704, respectively. Mutant 1, 2 and 3 displayed an OD50 significantly higher than the acceptor capsid (FIGS. 4A, B, C and F), whereas mutant 4 and 5 were totally comparable to AAV8 profile without any significant difference in OD50 (FIGS. 4D, E and F). All the mutants displayed an OD50 significantly lower than the donor capsids (FIG. 4F).

These results thus demonstrate that rational shuffling can be used as method to combine the capsid properties of multiple parental capsids. Furthermore, these results altogether demonstrate that rational shuffling can be used as method to transfer capsid properties from donor to acceptor capsid without alteration of acceptor capsid seroprevalence.

### Example 2: Production, In Vitro and In Vivo Testing of Hybrid Capsid from AAV8 with Single HVR Replacement

In order to better characterize the properties of the 12 HVRs from #704, the HVRs of AAV8 are substituted one by one with the corresponding HVR of wild type #704 capsid. The amino acid sequences of HVR2 and 4 of #704 are identical to AAV8, therefore 10 AAV8 capsids with a single HVR substitution are analyzed:
AAV8-mut.HVR1 (SEQ ID NO: 37) encoded by the polynucleotide of SEQ ID NO: 86;
AAV8-mut.HVR3 (SEQ ID NO: 38) encoded by the polynucleotide of SEQ ID N: 88;
AAV8-mut.HVR6 (SEQ ID NO: 39) encoded by the polynucleotide of SEQ ID NO: 90;
AAV8-mut.HVR7 (SEQ ID NO: 40) encoded by the polynucleotide of SEQ ID NO: 92;
AAV8-mut.HVR8 (SEQ ID NO: 41) encoded by the polynucleotide of SEQ ID NO: 94;
AAV8-mut.HVR9 (SEQ ID NO: 42) encoded by the polynucleotide of SEQ ID NO: 96;
AAV8-mut.HVR10 (SEQ ID NO: 43) encoded by the polynucleotide of SEQ ID NO: 98;
AAV8-mut.HVR11 (SEQ ID NO: 44) encoded by the polynucleotide of SEQ ID NO: 100; and
AAV8-mut.HVR12 (SEQ ID NO: 45) encoded by the polynucleotide of SEQ ID NO: 102.

Recombinant AAV vectors are produced by cloning the modified Cap genes in a plasmid suitable for vector production. A transgene expression cassette flanked by AAV2 ITRs and expressing a luciferase reporter gene is encapsidated in the so derived AAV vectors. Triple transfection of HEK293 cells is used to produce the vectors followed by immunoaffinity column purification. Vectors are tested in vitro in in cell lines and in primary cells obtained from a commercial source. In parallel, the vectors are tested in wild-type C57Bl6/J mice through intravenous injection of the different vectors at the dose of 1×10¹¹ vg/mice. Fifteen days post-injection, animals are sacrificed and the levels of expression of the transgene are measured in isolated tissues. The seroprevalence of mutant capsids is tested by ELISA as shown in EXAMPLE 1.

In all tested muscles, brain and spinal cord, all mutant capsids except AAV8-mut.HVR11 showed higher efficiency than AAV8 (FIG. 5). In particular, the luciferase activity of AAV8-mut.HVR3 and 12 was significantly higher than AAV8 in at least one muscle. Concerning CNS, AAV8-mut.HVR3, AAV8-mut.HVR9, AAV8-mut.HVR10 and AAV8-mut.HVR12 were significantly more efficient than AAV8 in spinal cord and/or brain. All mutants showed a seroprevalence significantly lower than donor capsid #704 and equivalent to acceptor capsid AAV8 (FIG. 6). The level of anti-AAV antibodies in AAV8-mut.HVR6 was significantly lower than acceptor capsid (OD50: 145 and 400 in mutant and AAV8, respectively), whereas AAV8-mut.HVR12 showed a low seroprevalence (OD50: 631) but still significantly higher than AAV8. These results suggest that the substitution of a single HVR can improve the acceptor tropism without altering its seroprevalence.

### Example 3: Production, In Vitro and In Vivo Testing of Hybrid Capsid from AAV8 with Different HVR5 from Wild Type AAV

The wild type capsids recently isolated in human liver (La Bella T et al., Gut., 2020, 69, 737-747.doi: 10.1136/gutjnl-2019-318281) represent the variability of AAV in a context of natural infection. These 59 capsids are characterized by specific amino acid variations involving also the HVR5. The alignment of wild type AAV capsids from two different genotypes, AAV2 and AAV2/13, AAV13 (GenBank accession number ABZ10812.1) and AAV2 (GenBank accession number YP_680426.1) allowed the identification of 19 unique HVR5 sequences including 4 from AAV2 serotype (wild-type AAV2; wild-type capsid #2102, #1343, #3013), 14 from AAV2/13 serotype (wild-type capsid #1704, #3086, #1591, #3142, #985, #M258, #1570, #2806, #2731, #1602, #667, #129, #217, #767) and 1 from AAV13 serotype (wild-type capsid #508). Similar to mutant 5 in EXAMPLE 1, new AAV8 mutants containing 12 different HVR5 substitutions are generated in order to characterize the properties of the new AAV mutants.

Mutant5-AAV2 (SEQ ID NO: 46) encoded by the polynucleotide of SEQ ID NO: 104, which comprises an HVR5 of SEQ ID NO: 187 encoded by the polynucleotide of SEQ ID NO: 201.

Mutant5-AAV13:
Mutant5-#508 (SEQ ID NO: 49) encoded by the polynucleotide of SEQ ID NO: 110), which comprises an HVR5 of SEQ ID NO: 186 encoded by the polynucleotide of SEQ ID NO: 200. SEQ ID NO: 186 is HVR5 sequence of AAV13.

Mutant5-AAV2/13:
Mutant5-#1704 (SEQ ID NO: 47) encoded by the polynucleotide of SEQ ID NO: 106, which comprises an HVR5 of SEQ ID NO: 176 encoded by the polynucleotide of SEQ ID NO: 190.
Mutant5-#3086 (SEQ ID NO: 48) encoded by the polynucleotide of SEQ ID NO: 108, which comprises an HVR5 of SEQ ID NO: 177 encoded by the polynucleotide of SEQ ID NO: 191.
Mutant5-#3142 (SEQ ID NO: 50) encoded by the polynucleotide of SEQ ID NO: 112, which comprises an HVR5 of SEQ ID NO: 178 encoded by the polynucleotide of SEQ ID NO: 192.
Mutant5-#M258 (SEQ ID NO: 51) encoded by the polynucleotide of SEQ ID NO: 114 which comprises an HVR5 of SEQ ID NO: 179 encoded by the polynucleotide of SEQ ID NO: 193.
Mutant5-#1570 (SEQ ID NO: 52) encoded by the polynucleotide of SEQ ID NO: 116, which comprises an HVR5 of SEQ ID NO: 180 encoded by the polynucleotide of SEQ ID NO: 194.
Mutant5-#2731 (SEQ ID NO: 53) encoded by the polynucleotide of SEQ ID NO: 118, which comprises an HVR5 of SEQ ID NO: 181 encoded by the polynucleotide of SEQ ID NO: 195.
Mutant5-#1602 (SEQ ID NO: 54) encoded by the polynucleotide of SEQ ID NO: 120, which comprises an HVR5 of SEQ ID NO: 182 encoded by the polynucleotide of SEQ ID NO: 196.
Mutant5-#667 (SEQ ID NO: 55) encoded by the polynucleotide of SEQ ID NO: 122, which comprises an HVR5 of SEQ ID NO: 183 encoded by the polynucleotide of SEQ ID NO: 197.
Mutant5-#129 (SEQ ID NO: 56) encoded by the polynucleotide of SEQ ID NO: 124), which comprises an HVR5 of SEQ ID NO: 184 encoded by the polynucleotide of SEQ ID NO: 198.
Mutant5-#767 (SEQ ID NO: 57) encoded by the polynucleotide of SEQ ID NO: 126), which comprises an HVR5 of SEQ ID NO: 185 encoded by the polynucleotide of SEQ ID NO: 199.

Mutant 5 (Example 1) comprises HVR5 from #704 which as the sequence SEQ ID NO: 175 encoded by the polynucleotide of SEQ ID NO: 189.

HVR5 from #704 (SEQ ID NO: 175) is present in other wild-type capsids of hybrid serotype 2/13 (#1010 (SEQ ID NO: 6); #2112, #1350, #668, #367, #1020, #1158, #2107 (SEQ ID NO: 11 to 17), #714 (SEQ ID NO: 19), #790, #976, #1286, #163, #685, #442, #2320 (SEQ ID NO: 22 to 29)).

HVR5 from AAV13 (SEQ ID NO: 186) is present in wild-type capsid #1024 (SEQ ID NO: 22) and #508 (SEQ ID NO: 9).

Recombinant AAV vectors are produced by cloning the modified Cap genes in a plasmid suitable for vector production. A transgene expression cassette flanked by AAV2 ITRs and expressing a luciferase reporter gene is encapsidated in the so derived AAV vectors. Triple transfection of HEK293 cells is used to produce the vectors followed by immunoaffinity column purification. Vectors are tested in vitro in in cell lines and in primary cells obtained from a commercial source. In parallel, the vectors are tested in wild-type C57Bl6/J mice through intravenous injection of the different vectors at the dose of 1×10¹¹ vg/mice. Fifteen days post-injection, animals are sacrificed and the levels of expression of the transgene are measured in isolated tissues. The seroprevalence of mutant capsids is tested by ELISA as shown in EXAMPLE 1.

All mutant capsids with HVR5 of AAV13 (Mutant5-#508) or hybrid AAV2/13 serotype showed higher efficiency than AAV8 in one or more of muscle, brain, and spinal cord. In particular, the luciferase activity of Mut5-#1704, Mut5-#3086 and Mut5-#M258 was significantly higher than AAV8 in at least one muscle. Mut5-#1704, Mut5-#3086 and Mut5-#3142 were significantly more efficient than AAV8 in spinal cord targeting. In contrast, mutant capsids with HVR5 of AAV2 serotype showed no improvement compared to AAV8 in all tested muscles, brain and spinal cord (FIG. 7).

These results suggest that AAV13 and AAV2/13 serotypes can be used as donor capsids for the substitution of the HVR5 of AAV8 using rational shuffling.

### Example 4: Production, In Vitro and In Vivo Testing of Hybrid Capsid from AAV8 with Different HVR5-8 from Wild Type AAV

Similar to mutant 4 in EXAMPLE 1, new AAV8 mutants containing the combinations of HVR5, 6, 7 and 8 naturally present in wild type AAV capsids are designed. The wild type capsids recently isolated in human liver (La Bella T et al., Gut., 2020, 69, 737-747.doi: 10.1136/gutjnl-2019-318281), AAV13 (GenBank accession numberABZ10812.1), AAV2 (GenBank accession number YP_680426.1) were multialigned allowing the identification of 27 unique combinations of HVR5, 6, 7 and 8 including 1 from AAV13 serotype (wild-type AAV13), 7 from AAV2 serotype (wild-type AAV2; #2497, #2102, #2087, #1449, #1343, #3013) and 19 from AAV2/13 serotype (#1704, #3086, #1024, #1591, #508, #3142, #2320, #1010, #985, #M258, #1570, #2806, #2731, #2112, #1602, #667, #129, #217, #767). 15 combinations were included in AAV8 capsid in order to characterize the properties of the new AAV mutants.
Mutant4-AAV13 (SEQ ID NO: 58) encoded by the polynucleotide of SEQ ID NO: 128);
Mutant4-AAV2 (SEQ ID NO: 59) encoded by the polynucleotide of SEQ ID NO: 130);
Mutant4-AAV2/13 serotype:
Mutant4-1704 (SEQ ID NO: 60) encoded by the polynucleotide of SEQ ID NO: 132);
Mutant4-3086 (SEQ ID NO: 61) encoded by the polynucleotide of SEQ ID NO: 134);
Mutant4-1024 (SEQ ID NO: 62) encoded by the polynucleotide of SEQ ID NO: 136);
Mutant4-508 (SEQ ID NO: 63) encoded by the polynucleotide of SEQ ID NO:138);
Mutant4-3142 (SEQ ID NO: 64) encoded by the polynucleotide of SEQ ID NO: 140);
Mutant4-2320 (SEQ ID NO: 65) encoded by the polynucleotide of SEQ ID NO: 142);
Mutant4-1010 (SEQ ID NO: 66) encoded by the polynucleotide of SEQ ID NO: 144);
Mutant4-M258 (SEQ ID NO: 67) encoded by the polynucleotide of SEQ ID NO: 146);
Mutant4-1570 (SEQ ID NO: 68) encoded by the polynucleotide of SEQ ID NO: 148);
Mutant4-1602 (SEQ ID NO: 69) encoded by the polynucleotide of SEQ ID NO: 150);
Mutant4-667 (SEQ ID NO: 70) encoded by the polynucleotide of SEQ ID NO: 152);
Mutant4-129 (SEQ ID NO: 71) encoded by the polynucleotide of SEQ ID NO: 154);
Mutant4-767 (SEQ ID NO: 72) encoded by the polynucleotide of SEQ ID NO: 156).

Mutant 4 (Example 1) comprises HVR5, HVR6, HVR7 and HVR8 from the capsid #704 (SEQ ID NO: 2). HVR5, HVR6, HVR7 and HVR8 from capsid #704 are present in other wild-type capsids of hybrid serotype 2/13 (#2112, #1350, #668, #367, #1020, #1158, #2107 (SEQ ID NO: 11 to 17), #714 (SEQ ID NO: 19), #790, #976, #1286, #163, #685, #442 (SEQ ID NO: 22 to 28)).

Recombinant AAV vectors are produced by cloning the modified Cap genes in a plasmid suitable for vector production. A transgene expression cassette flanked by AAV2 ITRs and expressing a luciferase reporter gene is encapsidated in the so derived AAV vectors. Triple transfection of HEK293 cells is used to produce the vectors followed by immunoaffinity column purification. Vectors are tested in vitro in in cell lines and in primary cells obtained from a commercial source. In parallel, the vectors are tested in wild-type C57Bl6/J mice through intravenous injection of the different vectors at the dose of 1×10¹¹ vg/mice. Fifteen days post-injection, animals are sacrificed and the levels of expression of the transgene are measured in isolated tissues. The seroprevalence of mutant capsids is tested by ELISA as shown in EXAMPLE 1.

The majority of mutant capsids with HVR5 to HVR8 of AAV2/13 serotype showed higher efficiency than AAV8 in muscle, brain, and/or spinal cord. Mut4-AAV13 and mut4-#M258 showed significantly higher luciferase activity than AAV8 in soleus and spinal cord, respectively. In contrast, mutant capsids with HVR5 of AAV2 serotype showed no improvement compared to AAV8 in all tested muscles and brain (FIG. 8).

These results suggest that the substitution of HVR5-8 of AAV8 with AAV13 or AAV2/13 serotype as donor capsids, can enhance muscle and/or CNS targeting the acceptor capsid.

### Example 5: Production, In Vitro and In Vivo Testing of Wild Type HVR5 on a Different Reference Capsid

The HVR5 of #704 is cloned in a different AAV reference capsid already used in gene therapy, AAV9 (GenBank Accession numbers: AY530579.1). AAV9-R5-704 (SEQ ID NO: 73) is encoded by the polynucleotide of SEQ ID NO: 158).

Recombinant AAV vectors are produced by cloning the modified Cap genes in a plasmid suitable for vector production. A transgene expression cassette flanked by AAV2 ITRs and expressing a luciferase reporter gene is encapsidated in the so derived AAV vectors. Triple transfection of HEK293 cells is used to produce the vectors followed by immunoaffinity column purification. Vectors are tested in vitro in in cell lines and in primary cells obtained from a commercial source. In parallel, the vectors are tested in wild-type C57Bl6/J mice through intravenous injection of the different vectors at the dose of 1×10¹¹ vg/mice. Fifteen days post-injection, animals are sacrificed and the levels of expression of the transgene are measured in isolated tissues. The seroprevalence of mutant capsids is tested by ELISA as shown in EXAMPLE 1.

The mutant capsid AAV9-R5-704 showed higher efficiency than AAV8 in muscle, brain, and spinal cord (FIG. 9). The mutant capsid AAV9-R5-704 showed a seroprevalence significantly lower than donor capsid #704 and equivalent to acceptor capsid AAV9 (FIG. 10).

These results show that the substitution of HVR5 using rational shuffling is a valuable method to improve muscle and/or CNS targeting of other acceptor capsids, like AAV9.

1-28. (canceled)

29. A method of preparation of a recombinant hybrid adeno-associated virus (AAV) capsid protein with improved tropism for muscle and/or central nervous system, comprising the steps of
a) providing at least two recombinant AAV capsid proteins from different AAV serotypes, an acceptor AAV capsid protein and at least one donor AAV capsid protein, wherein the donor AAV capsid serotype is AAV13 or hybrid AAV2/13;
b) replacing at least one hypervariable region (HVR) sequence chosen from HVR1 to HVR10 and HVR12 sequences of the acceptor AAV capsid protein with a different HVR sequence from the corresponding HVR of a donor AAV capsid protein, to obtain a recombinant hybrid AAV capsid protein with improved tropism for muscle and/or central nervous system compared to at least the parent acceptor AAV capsid protein.

30. The method of claim 29, wherein the acceptor AAV capsid serotype has a low seroprevalence and the donor AAV capsid serotype(s) has a higher seroprevalence than the acceptor AAV capsid serotype.

31. The method of claim 29, wherein the acceptor AAV capsid serotype is selected from the group consisting of: AAV8, AAV9, AAV5, AAV-LK03, AAVrh74, AAV9.rh74, AAV9.rh74-P1 and AAVrh10 and/or the donor AAV capsid serotype(s) is selected from the group consisting of AAV13 and the sequences SEQ ID NO: 2 to 30.

32. The method of claim 31, wherein the acceptor AAV capsid serotype is selected from the group consisting of: AAV8 and AAV9 and the donor AAV capsid serotype(s) is selected from the group consisting of AAV13 and the sequences SEQ ID NO: 2 to 30.

33. The method of claim 29, wherein the hybrid AAV capsid protein has a seroprevalence equivalent to the seroprevalence of the acceptor AAV capsid protein.

34. The method of claim 29, wherein the HVR sequence(s) of the donor AAV capsid protein and/or acceptor AAV capsid protein(s) are selected from the group consisting of an HVR1 sequence from positions 134 to 165, an HVR2 sequence from positions 176 to 192; an HVR3 sequence from positions 259 to 278; an HVR4 sequence from positions 379 to 395; an HVR5 sequence from positions 446 to 484; an HVR6 sequence from positions 490 to 500; an HVR7 sequence from positions 501 to 512; an HVR8 sequence from positions 514 to 529; an HVR9 sequence from positions 531 to 570; an HVR10 sequence from positions 576 to 613; and an HVR12 sequence from positions 705 to 736; the indicated positions being determined by alignment with SEQ ID NO: 1.

35. The method of claim 29, wherein step b) comprises replacing less than 8 HVR sequences such as up to 6 or up to 4 HVR sequences of the acceptor AAV capsid protein with different HVR sequence(s) from the corresponding HVR(s) of the donor AAV capsid protein(s).

39. The method of claim 29, wherein step b) comprises replacing at least HVR5 sequence of the acceptor AAV capsid protein with a different HVR5 sequence from the donor AAV capsid protein.

40. The method of claim 39, wherein said HVR5 sequence from the donor AAV capsid protein comprises a sequence selected from the group consisting of SEQ ID NO: 175 to 186.

41. The method of claim 39, wherein step b) comprises replacing HVR5 sequence alone or in combination with one or more or all of HVR6, HVR7 and HVR8 or with one or more or all HVR6, HVR7, HVR8, HVR9 and HVR10 of the acceptor AAV capsid protein.

42. The method of claim 29, wherein step b) comprises replacing any one of HVR1 to HVR10 and HVR12 sequence or any one of HVR3, HVR5, HVR9, HVR10 or HVR12 sequence of the acceptor AAV capsid protein with a different HVR sequence from the corresponding HVR of the donor AAV capsid protein.

43. A recombinant hybrid AAV capsid protein with improved tropism obtainable by the method of claim 29.

44. The recombinant hybrid AAV capsid protein of claim 43, which comprises an amino acid sequence selected from the group consisting of the sequences SEQ ID NO: 33 to 43, 45, 47 to 58 and 60 to 73 and the variant sequences having at least 85% identity with said sequences, and wherein the variant sequence has no mutations in at least the HVR sequences from the donor AAV capsid protein or all the HVR sequences.

45. A recombinant plasmid comprising a polynucleotide encoding in expressible form, a recombinant hybrid AAV capsid protein with improved tropism obtainable by the method of claim 29.

46. The recombinant plasmid of claim 45, which comprises a polynucleotide selected from the group consisting of the nucleotide sequences SEQ ID NO: 78, 80, 82, 84, 86, 88, 90, 92, 94, 96, 98, 102, 106, 108, 110, 112, 114, 116, 118, 120, 122, 124, 126, 128, 132, 134, 136, 138, 140, 142, 144, 146, 148, 150, 152, 154, 156 and 158.

47. A cell, stably transformed with a recombinant plasmid comprising a polynucleotide encoding, in expressible form, a recombinant hybrid AAV capsid protein with improved tropism obtainable by the method of claim 29.

48. An AAV vector particle packaging a gene of interest, which comprises at least one recombinant hybrid AAV capsid protein with improved tropism obtainable by the method of claim 29.

49. The AAV vector particle of claim 48, wherein the gene of interest is selected from the group consisting of: therapeutic genes; genes encoding therapeutic proteins or peptides such as therapeutic antibodies or antibody fragments and genome editing enzymes; and genes encoding therapeutic RNAs such as interfering RNAs, guide RNAs for genome editing and antisense RNAs capable of exon skipping.

50. A pharmaceutical composition comprising a therapeutically effective amount of AAV vector particle of claim 48, or cell stably transduced by said AAV vector particle.

51. A method of treatment of a muscle, central nervous system disease, and/or a neuromuscular genetic disease in a patient in need thereof, comprising administering to the patient a therapeutically effective amount of the pharmaceutical composition of claim 50.
